(19) United States
(12) Patent Application Publication
(10) Pub. No.: US 2024/0299940 A1
(43) Pub. Date: Sep. 12, 2024

(54) TARGET DETECTION SYSTEMS AND METHODS

(71) Applicant: DOTS Technology Corp., Natick, MA (US)

(72) Inventors: Adi Gilboa-Geffen, Wayland, MA (US); Renuka Babu Brown, Weston, MA (US)

(21) Appl. No.: 18/260,600

(22) PCT Filed: Jan. 6, 2022
(86) PCT No.: PCT/US22/11359

§ 371 Date: Jul. 6, 2023

Related U.S. Application Data
(60) Provisional application No. 63/134,225, filed on Jan. 6, 2021.

(51) Int. Cl. B01L 3/00; G01N 33/543

(52) U.S. Cl. CPC B01L 3/502738; G01N 33/54366; B01L 2200/04; B01L 2200/0689; B01L 2200/10; B01L 2300/042; B01L 2300/044; B01L 2300/0672; B01L 2300/0681; B01L 2300/12; B01L 2400/0644

(57) ABSTRACT

The present disclosure is drawn to devices and systems for target detection in samples, including food samples, clinical samples, environmental samples and veterinary samples. The target detection system includes a sampler, a disposable analysis cartridge and a detection device with an optimized optical system.

### CROSS REFERENCE TO RELATED APPLICATIONS

The present application claims priority to U.S. Provisional Application No. 63/134,225, filed Jan. 6, 2021, and entitled with “TARGET DETECTION SYSTEMS AND METHODS”; the contents of which are incorporated herein by reference in their entirety.

### FIELD OF THE DISCLOSURE

The present disclosure is drawn to portable devices and systems for target detection in samples. The disclosure also provides methods for detecting the presence and/or absence of a molecule of interest in a sample.

### BACKGROUND OF THE DISCLOSURE

Antibodies are specific to antigens and widely used to detect antigens for may purposes such as clinical diagnosis of diseases. Antibodies have been used for target detection in various fields including immunoassays and immunosensors. These antibody-based assays have extensive applications in all areas of clinical and pharmaceutical chemistry as well as food industry, veterinary care and environmental analysis.

The present disclosure provides a portable assembly and a device for fast and accurate detection of a molecule of interest in a sample using an antibody chip. The antibodies, as detection agents, specifically bind to the molecule of interest, forming antibody complexes which are them detected and measured using an integrated detection system of the present disclosure. The detection system may comprise a separate sampler, disposable cartridges/vessels for processing the sample and implementing the detection assay, and a detector unit including an optical system for operating the detection and detecting the reaction signal. The detection agents (e.g., antibodies) may be integrated into the disposable cartridges of the present disclosure. The cartridges, detection agents and the detection sensors may also be used in other detection systems. Antibodies specific to molecules of interest (e.g., allergen proteins) may also be used in the present detection systems. Such devices may be used by consumers in non-clinical settings, for example in the home, in restaurants, school cafeteria and food processing facilities.

### SUMMARY OF THE DISCLOSURE

The present disclosure provides systems, devices, disposable cartridges/vessels, optical systems and methods for use in detection of a molecule of interest in various types of samples including food samples, clinical samples, environmental and veterinary samples. The detection devices and systems are portable and handheld.

An aspect of the present disclosure is an assembly for detecting a molecule of interest of in a sample such as a food sample, a clinical sample, an environmental and a veterinary sample. The assembly comprises an analytical cartridge configured to accept the sample for processing to a state permitting the molecule of interest to engage in an interaction with antibody-based detection agent. The assembly includes a detector unit configured to accept the analytical cartridge in a configuration which permits a detection mechanism housed by the detector unit to detect the interaction of the molecule of interest with antibodies. The interaction triggers a visual indication on the detector unit that the molecule of interest is present or absent in the sample. The detector unit may be removably connected to the analytical cartridge.

In some embodiments, the assembly further comprise a separate sampler configured to collect a sample for detection of the molecule of interest in the sample. In some embodiments, the sampler is a sampling corer. The corer may be operatively connected to the analytical cartridge to transfer the collected sample to the cartridge.

In some embodiments, the analytical cartridge is disposable, and configured to detect one particular molecule of interest. In other embodiments, the analytical cartridge may be configured to detect a plurality of molecules of interest in a sample.

In some embodiments, the analytical cartridge comprises a homogenizer configured to produce a homogenized sample, thereby releasing the molecule of interest from a matrix of the sample into an extraction buffer. The analytical cartridge also comprises a first conduit to transfer the homogenized sample with or without the detection agent through a filter system to provide a filtrate containing the molecule of interest, or the complexes of the molecule of interest and the detection agent, and a second conduit to transfer the filtrate, making the filtrate to be contacted with the detection agent (e.g., antibodies). The first and second conduits comprise a plurality of fluidic paths connecting different parts of the conduits from transferring the processed sample, buffers, filtrate, detection agents, waste and other fluids.

In some embodiments, the analytical cartridge may further comprise a rotary valve system providing a mechanism for controlling the transfer of the sample and other fluidic components (e.g., buffers, filtrate, and waste) within the analytical cartridge. The rotary valve switching system may be further configured to provide a closed position to prevent fluid movement in the analytical cartridge.

In some embodiments, the homogenizer and the rotary valve system may be powered by motors located in the detector unit when the analytical cartridge is accepted by the detector unit.

In some embodiments, the analytical cartridge comprises a plurality of chambers. The chambers are separate but connected for operation. As a non-limiting example, the analytical cartridge may include a sample processing chamber, a detection chamber, a waste chamber, and optionally a buffer chamber. In some embodiments, the analytical cartridge may further comprise a separate filtrate chamber to hold the filtrate and optionally further concentrate the filtrate prior to the transfer to the detection chamber. In some examples, the detection chamber comprises a detection sensor and an optical window. The detection mechanism of the detector unit analyzes the detection reaction through the optical window to identify the interaction of the molecule of interest with the detection agent in the detection chamber.

In some embodiments, the detection sensor is a transparent substrate which includes a plurality of fluidic channels and a detector chip area. The substrate is also referred to as a chipannel, wherein the fluidic channels and the detector chip area are connected. In some examples, the chipannel is a plastic substrate.

In some embodiments, the detector chip area within the chipannel comprises at least one reaction panel and at least one control panel. In other embodiments, the detector chip area within the chipannel may comprise one reaction panel and two control panels. In other embodiments, the chipannel may comprise a plurality of reaction panels and a plurality of control panels. Optionally, the detector chip area further comprises one or more fiducial spots that guide image processing by an imaging mechanism (e.g., a camera) of the detector unit. Any suitable fiducial object may be spotted as a fiducial marker for reference.

In some embodiments, the detector chip area within the chipannel comprises an antibody immobilized on the reaction panel. The detector chip area within the chipannel may further include an optically detectable control antibodies immobilized on the control panel(s), for normalization of signal output measured by the detection mechanism.

The analytical cartridge may further include a chamber storing wash buffer for washing the detection chamber and a waste chamber for accepting outflow contents of the detection chamber after washing. In some embodiments, the series of bridging fluid conduits may comprise: (a) a fluid connection between the wash buffer chamber and the detection chamber; and (b) a fluid connection between the detection chamber and the waste chamber.

In some embodiments, the filter in the analytical cartridge is a filter assembly comprising a bulk filter and a membrane filter. The bulk filter may comprise a gross filter and a depth filter. In some embodiments, the filter assembly may further comprise a filter cap that can lock the rotary valve.

In some embodiments, the analytical cartridge may comprise a data chip unit configured for providing the cartridge information.

In an embodiment an analytic cartridge for detecting a molecule of interest in a sample comprising a first compartment with a homogenizer for receiving a sample and processing the sample. The homogenizer configured to produce a homogenized sample, thereby releasing the molecule of interest from a matrix of the sample into an extraction buffer in the presence of the detection agent and permitting the molecule of the interest in the sample to engage in the interaction with the detection agent. The cartridge includes a lid covering the cartridge, and the lid comprises at least one aperture opening into the first compartment, a cap rotatably connected to the lid, wherein the cap is capable of rotating from a first position to a second position, a seal on the at least one aperture creating a pocket between the seal and the cap, a homogenization accelerator positioned in the pocket when the cap is in a first position, and wherein when the cap is rotated to the second position the homogenization accelerator is released into the first compartment. The cartridge includes a conduit to transfer the homogenized sample and detection agent through a filter system to provide a filtrate containing the molecule of interest and the detection agent. The cartridge includes a second compartment for contacting the filtrate containing the molecule of interest and the detection agent with detection probes; the second compartment comprising a transparent substrate that comprises fluidic channels and a detection chip area with a detection probe immobilized thereon, the detection probe configured to engage in a probe interaction with the detection agent, wherein the interaction of the molecule of interest with the detection agent prevents the detection agent from engaging in the probe interaction with the detection probe.

The cartridge also includes a rotary valve system configured to regulate the transfer of the homogenized sample and detection agent through the filter system, of the filtrate to the second compartment, and of wash buffer to the second compartment and outflow contents from the second compartment to a waste chamber, a compartment for holding wash buffer for washing the detection area, and a waste chamber for accepting outflow contents of the detection chamber. The at least one aperture further includes a second aperture opening into the first compartment.

The cap further includes a port which, when the cap is in the first position, co-localizes with the second aperture; the second aperture containing a breakable seal facing the first compartment. When the cap is in the second position, the second aperture is covered by the cap and sealed by a movable cover. The cartridge may be used in combination with a detector device comprising an external housing configured for providing support for the components of the detection device. The components integrated for operating a detection test comprising an assembly lid capable of measuring the weight, mass, or volume of a sample, a motor for driving and controlling the sample homogenization, a motor for controlling a valve system, a pump for driving and controlling fluidic flow, an optical system for detecting fluorescence signals, means for converting and digitizing the fluorescence signals, a display window for receiving the detected signals and indicating the presence and/or absence of the target in the test sample, and a power supply.

Another aspect of the disclosure includes a test cup assembly for processing a sample to a state permitting detection of a molecule of interest in the sample comprising a top cover for sealing the test cup and providing an identification label, the top cover further comprising: a movable cap, the movable cap having a lancing element, and a homogenization accelerator, which is secured in a pocket bounded by the cap and a sealed aperture on the top cover. The test cup includes a body part for receiving and processing the sample to a state permitting the molecule of interest in the sample to engage in an interaction with a detection agent. The body part comprises a first compartment with a homogenizer for homogenizing the sample to extract the molecule of interest using an extraction buffer. The test cup includes a conduit for transferring the homogenized sample containing the molecule of the interest and detection agent through a filter system to provide a filtrate containing the molecule of interest, a chamber for holding wash buffer, a waste chamber for receiving and storing the outcome contents after washing the molecule of interest and the detection agent, and a rotary valve system for controlling the fluid movement inside the test cup assembly. The test cup includes a transparent substrate comprising a plurality of fluidic channels and a detection area with the detection agent immobilized thereon. The test cup also includes a bottom cover for scaling the test cup and providing an interface to connect the test cup to a detector unit for operating the detection. The bottom cover comprising a transparent window that is aligned with the detection area of the transparent substrate upon assembly of the test cup.

In some embodiments, the disposable analytical cartridge comprises a lid with a movable cap, the movable cap having a lancing element, and a homogenization accelerator, which is secured in a pocket bounded by the cap and a seal on the lid. The movable cap is rotatably secured to the lid, the lid comprising an aperture opening into the homogenization chamber, the pocket being adjacent with the aperture. Movement of the movable cap from a first position to a second position causes the lancing element to lance the seal allowing the homogenization accelerator to enter the homogenization chamber.

In some embodiments, the assembly of the present disclosure comprises a detector unit that is operatively connected to an analytical cartridge. In some embodiments, the detector unit of the assembly comprises a detection mechanism to measure detection signals, i.e., the interaction between the target and the detection agent. As a non-limiting example, the detection mechanism is an imaging system, such as a camera for fluorescence imaging.

In some embodiments, the detector unit of the assembly comprises an external housing that provides support for the components integrated for operating a detection reaction and measuring detection signals, of the detector unit and for accepting the analytical cartridge. In accordance with the present disclosure, the components for operating a detection reaction and measuring detection signals include motors for driving and controlling the homogenization, and controlling the rotary valve; pump driving and controlling the fluidic flow of the processed sample, the filtrate, buffers and waste in the compartments of the analytical cartridge; an optical system for detecting and visualizing a detection result; and a display window.

In some embodiments, the optical system may comprise excitation optics and emission optics and an optical reader. The optical system is modified for detecting signals from the detector chip area of the chipannel within the cartridge.

In other embodiments, the optical system may comprise a camera sensor (e.g., a CCD camera and a sCMOS camera) to generate images of a detection reaction of the detector chip area of the chipannel. The images are then processed to indicate the detection results.

In some embodiments, the detection device includes a frame attachable to the housing a base attached to the frame and a cover connected to the frame; wherein the cover includes a measurement device adjacent thereto and above the base. The measurement device is capable of detecting and measuring the weight, mass, or volume of the sample when the sample is placed on the cover. The measurement device is a strain gauge.

In some embodiments, the detection assembly may comprise a user interface that may be accessed and controlled by a software application. The software may be run by a software application on a personal device such as a smartphone, a tablet computer, a personal computer, a laptop computer, a smartwatch, and/or other devices. In some embodiments, the personal device runs on iOS or Android software. In some cases, the software may be run by an internet browser. In some embodiments, the software may be connected to a remote and localized server referred to as the cloud. The personal device and software may record test results and allow for community interaction. The interaction may include a physician being able to view the data and usage of the device by a patient. The interaction may also include a parent or family member being able to view the date and usage by a child or other family member.

An aspect of the disclosure includes an assembly for detecting a molecule of interest in a sample comprising a sample processing cartridge having a homogenization chamber configured to accept the sample for processing to a state permitting the molecule of interest to engage in an interaction with a detection agent. The cartridge comprises a lid, a movable cap having a lancing element, and a homogenization accelerator, which is secured in a pocket bounded by the cap and a seal on the lid. The assembly also includes a detector unit configured to accept the sample processing cartridge in a configuration which permits a detection mechanism housed by the detector unit to detect the interaction of the molecule of interest with the detection agent, wherein the interaction triggers a visual indication on the detector unit that the molecule of interest is detected. The visual indication is by processing images capturing the interaction of the molecule of interest with the detection agent. The movable cap is secured to the lid and further comprises at least one aperture opening into the homogenization chamber. The pocket of the assembly is co-located with the at least one aperture. Movement of the movable cap causes the lancing element to lance the seal allowing the homogenization accelerator to enter the homogenization chamber. The at least one aperture further includes a second aperture opening into the homogenization chamber. The cap further includes a port which, in a first position of the cap, co-localizes with the second aperture; the second aperture containing a breakable seal facing the homogenization chamber. In a second position of the cap the second aperture is covered by the cap and sealed by a movable cover.

The assembly further comprises an assembly lid capable of measuring the weight, mass, or volume of a sample. The assembly lid further comprises a frame, a base attached to the frame, and a cover connected to the frame. The cover includes a measurement device adjacent thereto and above the base, whereby the measurement device is capable of detecting and measuring the weight, mass, or volume of the sample when the sample is placed on the cover. The measurement device is a strain gauge or a load gauge.

In a non-limiting embodiment of the present disclosure, a detection assembly comprises an analytical cartridge that is configured to be a disposable test cup or cup-like container, a detector unit comprising a docket for accepting the test cup, and an optional sampler. The disposable test cup or cup-like container may be constructed as an analytical module in which a sample is processed and a molecule of interest in the test sample (e.g., an allergen) is detected through the interaction with a detection agent.

In some embodiments, the disposable test cup or cup-like container comprises a top cover configured to accept the sample and to seal the cup or cup-like container wherein the top cover includes a port for accepting the sample and at least one breather filter that allows air in; a body part configured to process the sample to a state permitting the molecule of interest to engage in an interaction with the detection agent, and a bottom cover configured to connect to the cup body part thereby forming a detection chamber with an optical window at the bottom of the test cup, and to provide the connecting surface to a detector unit. The exterior of the bottom cover comprises a plurality of ports for connecting a plurality of motors located in the detector unit to operate the homogenizer, the rotary valve system and the flow of the fluids. The optical window of the detection chamber is connected to the detection mechanism in the detector unit.

In some embodiments, the test cup or cup-like container further comprises a detection sensor such as a transparent substrate with detection agents immobilized thereon. The transparent substrate is a chipannel comprising a detection chip area with antibodies immobilized thereon and fluidic paths.

In one non-limiting embodiment of the present disclosure, the disposable test cup comprises (a) a first compartment with a homogenizer for receiving a sample and processing the sample: the homogenizer configured to produce a homogenized sample, thereby releasing the molecule of interest from a matrix of the sample into an extraction buffer in the presence of the detection agent and permitting the molecule of the interest in the sample to engage in the interaction with the detection agent; (b) a second compartment for contacting the filtrate containing the molecule of interest and the detection agent with detection probes; the second compartment comprising a chipannel that comprises a plurality of fluidic channels and a detection chip area with the detection probes immobilized thereon; (c) a conduit to transfer the homogenized sample and detection agent through a filter system to provide a filtrate containing the molecule of interest and the detection agent; (d) a rotary valve system configured to regulate the transfer of the homogenized sample and detection agent through the filter system, of the filtrate to the second compartment, and of wash buffer to the second compartment and outflow contents from the second compartment to a waste chamber; (e) a compartment for holding wash buffer for washing the detection area; and (f) a waste chamber for accepting outflow contents of the detection chamber. In some examples, the detection probe is configured to engage in a probe interaction with the detection agent, wherein the interaction of the molecule of interest with the detection agent prevents the detection agent from engaging in the probe interaction with the detection probe. The fluidic paths within the chipannel transfer the filtrate, making the filtrate to be contacted with the detection probe immobilized on the chip area, and transfer the outflow contents to the waste chamber.

In some embodiments, the cup top cover further comprises a layer for providing an identification label.

In some embodiments, the parts of the disposable test cup are molded together forming an analytic module.

### BRIEF DESCRIPTION OF THE DRAWINGS

FIG. 1 is a perspective view of an embodiment of a detection system in accordance with the present disclosure comprising a detection device 100 having an external housing 101 and a port or receptacle 102 configured for holding the disposable cartridge 300, a separate food corer 200 as an example of the sampler, a disposable test cup 300 as an example of the analytical cartridge. Optionally, a lid 103, execution/action button 104 that allows a user to execute a target detection testing and a USB port 105 may be included.

FIG. 2A is an exploded perspective view of one embodiment of the food corer 200 as an example of the sampler.

FIG. 2B is a perspective view of the sampler assembly 200.

FIG. 3A is a perspective view of an embodiment of a disposable test cup 300, comprising a cup top 310, a cup body 320 and a cup bottom 330.

FIG. 3B is a cross-sectional view of the test cup 300, illustrating features inside the cup 300.

FIG. 3C is an exploded view of the disposable test cup 300.

FIG. 3D is a top (left panel) perspective view and a bottom (right panel) perspective view of the top cover 312.

FIG. 3E is an exploded view of the cup top lid 311.

FIG. 3F is a top perspective view (left panel) and a bottom perspective view (right panel) of the cup body 320.

FIG. 3G is a bottom perspective view of the bottom of the upper housing 320a (upper panel) shown in FIG. 3C and a top perspective view of the inside of the outer housing 320b (lower panel) shown in FIG. 3C.

FIG. 3H is a bottom perspective view (left panel) and a top perspective view (right panel) of the cup bottom cover 337.

FIG. 3I is a bottom perspective view of the cup bottom surface after assembling the bottom 330 and the cup body 320.

FIG. 4A is an exploded view of one embodiment of the filter assembly 325.

FIG. 4B is a cross-sectional perspective view of one embodiment of the filtrate chamber 322 comprising a filter bed chamber 431 for placement of the filter assembly 325, a collection gutter 432 and a filtrate collection chamber 433.

FIG. 5A is a perspective view of an alternative embodiment of the cup 300.

FIG. 5B is an exploded view of the disposable test cup 300 of FIG. 5A (the filter 325 not shown).

FIG. 5C is a cross sectional perspective view of the cup 300 of FIG. 5A.

FIG. 6A is an exploded view of an alternative embodiment of the cup 300.

FIG. 6B is a top perspective view (right panel) and a bottom perspective view (left panel) of the cup body 320 of FIG. 6A.

FIG. 6C is a bottom perspective view of the cup bottom 337 and the bottom of the cup body 320 of FIG. 6A.

FIG. 6D is an alternative embodiment of the filter assembly 325.

FIG. 6E is a cross-sectional view of the filter cap 621 when is assembled with the rotary valve 350.

FIG. 6F is a perspective view of the rotary valve 350 (upper panel) and a bottom perspective view of the bottom of the rotary valve 350 (lower panel).

FIG. 6G is a bottom perspective view (upper panel) and atop perspective view (lower panel) of the cup bottom cover 337 shown in FIG. 6A.

FIG. 7A is an exploded view of an alternative embodiment of the cup 300; the cup 300 comprises a chipannel 710.

FIG. 7B is a perspective view of the chipannel 710 shown in FIG. 7A.

FIG. 7C is a bottom perspective view of the chipannel 710.

FIG. 7D is a bottom perspective view of an alternative embodiment of the chipannel 710.

FIG. 7E is exploded view of an alternative embodiment of the cup 300.

FIG. 7F is an alternative embodiment of the cup body in which the filter gasket 623 is overmolded to the cup body.

FIG. 7G is an alternative embodiment of the rotary valve 350 shown in FIG. 7E.

FIG. 7H is a cross-sectional view of the cup body 320 shown in FIG. 7E, showing the overmolded seal 713 to combine several parts into a single part.

FIG. 7I is an alternative embodiment of the cup bottom cover 337 with compression coil springs 721.

FIG. 7J is perspective views of the cup bottom cover 337 shown in FIG. 7I, demonstrating the compression coil springs 721 at the bottom.

FIG. 7K is a perspective view of the sacrificial weld bead material 722 in the bottom of the cup body 320 shown in FIG. 7E.

FIG. 8A is a top perspective view of the cup body 320 showing features relating to homogenization, filtration (F), wash (W1 and W2) and waste.

FIG. 8B is a scheme showing the positions of the rotary valve 350 during the sample preparation and sample washes.

FIG. 8C is a diagram displaying the fluid flow inside the cup 300.

FIG. 9A is a perspective view of the device 100.

FIG. 9B is a top perspective view of the device 100 in the absence of the lid 103.

FIG. 10A is a longitudinal cross-sectional view of the device 100.

FIG. 10B is a lateral cross-sectional view of the device 100.

FIG. 11A is a valve motor 1020 and associated components for controlling the operation of the rotary valve 350.

FIG. 11B is a top perspective view of the output coupling 1020 associated with the motor.

FIG. 12A is a top perspective view of one embodiment of the optical system 1030.

FIG. 12B is a side view of the optical system 1030 of FIG. 12A.

FIG. 13A is an illustration of a chip sensor 333 displaying the test area and control areas.

FIG. 13B is a top view of the optical system 1030 and chip 333 showing reflections providing fluorescence measurements of the chip 333.

FIG. 13C is a perspective view of another embodiment of the chip senor 333 or the sensing area 333′ of the chipannel 710 displaying one reaction panel 1312, one control panel 1313 and two fiducial panels 1311.

FIG. 13D shows an exemplary pattern of the probes in the reaction panel and control panel of the detection area 333′ of the chipannel 710.

FIG. 14A shows the optical assembly 1030 in a straight mode.

FIG. 14B shows the optical assembly 1030 in a folded mode.

FIG. 14C is a cross-sectional perspective view of one end of the device 100 (right side of FIG. 10B) showing emission optics 1420 including lenses 1421, 1423 and filters 1422a and 1422b placed in the stepped bore 1480 in the device 100.

FIG. 15A is a perspective view of another embodiment of the optical system 1030 comprising an excitation optics 1510, an emission optics 1520 and a camera-based detector 1530.

FIG. 15B is a cross sectional view of the optical components of FIG. 15A as the optical system is configured inside the detection device 100.

FIG. 16A shows an expanded view of an embodiment with a rotatable cap 1700 with bead 1703.

FIG. 16B shows a cross-section of the assembled embodiment in FIG. 17A.

FIG. 16C is a side plan view of the cross-section in FIG. 17B.

FIG. 17A is an expanded view of an embodiment with an integrated 1800 lid with a scale.

FIG. 17B is a side plan view of the device in FIG. 18A.

FIGS. 18A-B is a view of the sensor chip printed with antibodies.

### DETAILED DESCRIPTION OF THE DISCLOSURE

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Unless defined otherwise, all technical and scientific terms used herein have the same meaning as commonly understood by one of ordinary skill in the art to which this disclosure belongs. In the case of conflict, the present description will control.

The present disclosure provides detection assemblies and systems that can specifically detect low concentrations of targets in a variety of samples, such as allergens in food samples, and diagnostic markers in clinical samples. The detection system and/or device of the present disclosure is a miniaturized, portable, and hand-held product, which is intended to have a compact size which enhances its portability and discreet operation. A user can carry the detection system and device of the present disclosure and implement a rapid and real-time test of the presence and/or absence of one or more allergens in a food sample, prior to consuming the food. The detection system and device, in accordance with the present disclosure, can be used by a user at any location, such as at home or in a restaurant. The detection system and/or device displays the test result as a standard readout and the detection can be implemented by any user following the simple instructions on how to operate the detection system and device. A specific utility of this detection system is the ease and rapidity of the system. The detection systems and assemblies of the present disclosure may also be used to detect any molecule of interest (i.e., any target) in a sample in general; the molecule of interest may be a protein or a variant thereof, a nucleic acid molecule (e.g., a DNA or RNA molecule) or a variant thereof, a lipid, a sugar, a small molecule, or a cell.

In some embodiments, the detection system is constructed for simple, fast, and sensitive one-step execution from the introduction of the sample to the system. The system may complete a detection test in less than 10 minutes, less than 9 minutes, less than 8 minutes, less than 7 minutes, less than 6 minutes, less than 5 minutes, or less than 4 minutes, or less than 3 minutes, or less than 2 minutes, or less than 1 minute. In some examples, the detection may be completed in approximately 60 seconds, 55 seconds, 50 seconds, 45 seconds, 40 seconds, 35 seconds, 30 seconds, 25 seconds, 20 seconds, or 15 seconds.

In accordance with the present disclosure, the detection system may involve a mechatronic construction process integrating electrical engineering, mechanical engineering and computing engineering to implement and control the process of a target detection test, including but not limiting to, rechargeable or replaceable batteries, motor drivers for processing the test sample, pumps for controlling the flow of the processed sample solutions and buffers within the cartridge, printed circuit boards, and connectors that couple and integrate different components for a fast allergen testing. The detection device of the present disclosure also includes an optical system which is configured for detection of the presence and concentration of a molecule of interest in a test sample and conversion of detection signals into readable signals; and a housing which provides support for other parts of the detection device and integrates different parts together as a functional product.

In some embodiments, the detection system is constructed such that disposable analytical cartridges (e.g., a disposable test cup or cup-like container), unique to one or more specific molecules of interest (e.g., allergens), are constructed for receiving and processing a test sample and implementing the detection test, in which all the solutions are packed. Therefore, all the solutions may be confined in the disposable analytical cartridges. As a non-limiting example, a disposable peanut test cup may be used to detect peanut in any food sample by a user and discarded after the test. This prevents cross-contamination when different allergen tests are performed using the same device. In some embodiments, a separate sampler for collecting a test sample is provided.

In accordance with the present disclosure, the disposable analytical cartridge comprises sensor chips printed with detection agents such as antibodies or variants thereof, nucleic acid molecules or variants thereof, and small molecules. In some embodiments, the detection agents are antibodies or variants thereof. In some embodiments, the detection agents may be attached to a solid substrate such as the surface of a magnetic particle, silica, agarose particles, polystyrene beads, a glass surface, a plastic chip, a microwell, a chip (e.g., a microchip), or the like. It is within the scope of the present disclosure that such detection agents and detection probes can also be integrated into any suitable detection systems and instruments for similar purposes.

### Detection Assemblies and Systems

In accordance with the present disclosure, a detection system or assembly for implementing a detection test of a molecule of interest in a sample comprises at least one disposable analytical cartridge for processing the sample to a state permitting the molecule of interest to engage in an interaction with a detection agent such as aptamer or antibody, and a detector unit for detecting and visualizing the result of the detection (i.e., the interaction between the molecule of interest and the detection agent). Optionally, the detection system may further comprise at least one sampler for collecting a test sample. The sampler can be any tool that can be used to collect a portion of a test sample, e.g., a spoon. In some aspects, a particularly designed sampler may be included to the present detection system as discussed hereinbelow. The exemplary embodiments described below illustrate such detection systems and assemblies for detecting an allergen in a sample.

In general, the analytical cartridge is configured to accept the sample for processing to a state permitting the molecule of interest to engage in an interaction with a detection agent. The detector unit is configured to accept the analytical cartridge in a configuration which permits a detection mechanism housed by the detector unit to detect the interaction of the molecule of interest with the detection agent. The interaction triggers a visual indication on the detector unit that the molecule of interest is present or absent in the sample. The detector unit may be removably connected to the analytical cartridge.

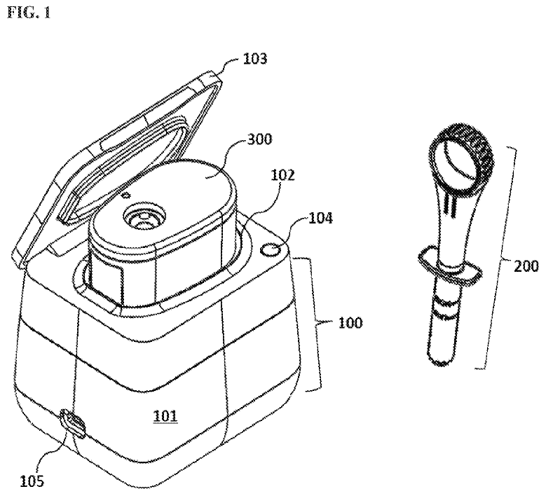

As shown in FIG. 1, an embodiment of the detection system or assembly of the present disclosure comprises a detection device 100 configured for processing a test sample, implementing a detection test, and detecting the result of the detection test, a separate sampling corer 200 as an example of the sampler, and a disposable test cup 300 as an example of the analytical cartridge. The detection device 100 includes an external housing unit 101 that provides support to the components of the detection device 100. A port or receptacle 102 of the detection device 100 is constructed for docking the disposable test cup 300 and a lid 103 is included to open and close the instrument. The external housing unit 101 also provides surface space for buttons that a user can operate the device. An execution/action button 104 that allows a user to execute an allergen detection testing and a USB port 105 may be included. Optionally a power plug (not shown) may also be included. During the process of implementing an allergen detection test, the sampling corer 200 with a sample contained therein is inserted into the disposable test cup 300 and the disposable test cup 300 is inserted into the port 102 of the detection device 100 for detection.

### Sampler

Collecting an appropriately sized sample is an important step for implementing allergen detection testing. In some embodiments of the present disclosure, a separate sampler for picking up and collecting test samples is provided. In one aspect, a coring-packer-plunger concept for picking up and collecting a sample is disclosed herein. Such mechanism may measure and collect one or several sized portions of the test sample and provide pre-processing steps such as cutting, grinding, abrading and/or blending, for facilitating the homogenization and extraction or release of allergen proteins from the test sample. The sampler may be operatively connected to the analytical cartridge and the detector unit for transferring a test sample to the cartridge for sample processing. According to the present disclosure, a separate sampling corer 200 is constructed for obtaining different types of samples and collecting an appropriately sized portion of a test sample. In one example, the sample is a liquid sample. In another example, the sample is a solid sample. It may be a raw sample or a preprocessed sample.

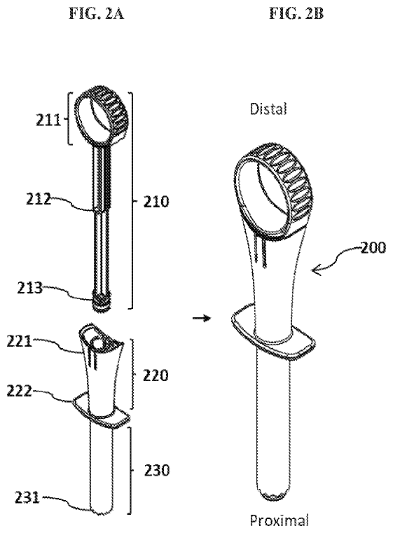

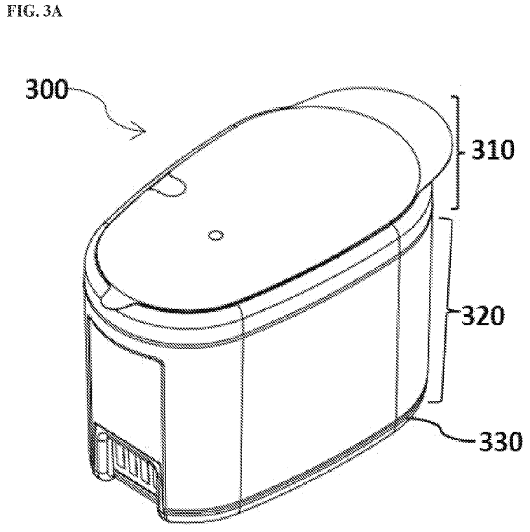

As shown in FIG. 2A, an embodiment of the corer 200 may comprise three parts: a plunger 210 at the distal end, a handle 220 configured for coupling a corer 230 at the proximal end. The plunger 210 has a distal portion provided with a corer top grip 211 (FIG. 2A) at the distal end, which facilitates maneuverability of the plunger 210 up and down, a plunger stop 212 in the middle of the plunger body, and a seal 213 at the proximal end of the plunger body. The handle 220 may comprise a snap fit 221 at the distal end and a projecting flat collar at the proximal end connecting to the corer 230. In one embodiment, the projecting flat collar comprises a flange 222 as shown in FIG. 2A. The corer 230 may comprise a proximal portion provided with a cutting edge 231 at the very proximal end (FIG. 2A). The corer 230 is configured for cutting and holding the collected sample to be expelled into the disposable test cup 300.

In some embodiments, the distal end of the plunger 210 may comprise a push plate. The plate may be a flat plate, in any shape. In one preferred embodiment, the push plate may be in a rounded square shape with a flared surface. Additionally, the rounded square shape provides an anti-roll feature when the sampler 200 is on a flat surface. This feature also can keep the collected sample inside the corer 230 (i.e., the sample area) from contacting an outside surface (e.g., a table when the sampler is lying on the table).

In some embodiments, the projecting flat collar may be configured as a small circular ring, a rib, or the like. This projection may prevent fingers from sliding down into the sample area and provide tactile orientation as well. As a non-limiting example, the projecting flat collar is a small circular ring.

In one embodiment, the plunger 210 may be inserted inside the corer 230, where the proximal end of the plunger 210 may protrude from the corer 230 for directly contacting a test sample, and together with the cutting edge 231 of the corer 230, picking up a sized portion of the test sample (FIG. 2B). In accordance with the present disclosure, the plunger 210 is used to expel sampled food from the corer 230 into the disposable test cup 300, and to pull certain foods into the corer 230 as well, such as liquids and creamy foods. The feature of the plunger stop 212, through an interaction with the snap fit 221, may prevent the plunger 210 from being pulled back too far or out of the corer body 230 during sampling. The seal 213 at the very proximal end of the plunger 210 may maintain an air-tight seal in order to withdraw liquids into the corer 230 by means of pulling the plunger 210 back. In some embodiments, the plunger 210 may be provided with other types of seals including a molded feature, or a mechanical seal. The handle 220 is constructed for a user to hold the coring component of the sampler 200. For example, the skirt 222 gives the user means for operating the food sampler 200, pushing down the corer 230 and driving the corer 230 into the food sample to be collected.

In some embodiments, the plunger 210 may comprise markings to provide additional guidance to the user, indicating the position of the plunger inside the corer and its position relevant to the minimal and maximal sampling lines. In some embodiments, the lines indicating the minimal and maximal amounts of the sample to be collected are added to the exterior of the corer 230. A user can correct the size of the sampling compartment by adjusting the minimal and maximal lines.

In some embodiments, the cutting edge 231 may be configured for pre-processing the collected sample, allowing the collected sample to be cored in a pushing, twisting and/or cutting manner. The cutting edge 231 may cut a portion from the test sample. As some non-limiting examples, the cutting edge 231 may be in a flat edge, a sharp edge, a serrated edge with various numbers of teeth, a sharp serrated edge and a thin wall edge. In other aspects, the inside diameter of the corer 230 varies, ranging from about 5.5 mm to 7.5 mm. Preferably the inside diameter of the corer 230 may be from about 6.0 mm to about 6.5 mm. The inside diameter of the corer 230 may be 6.0 mm, 6.1 mm, 6.2 mm, 6.3 mm, 6.4 mm, 6.5 mm, 6.6 mm, 6.7 mm, 6.8 mm, 6.9 mm, or 7.0 mm. The size of the corer 230 is optimized for a user to collect a right amount of the test sample (e.g., 1.0 g to 0.5 g).

The parts of the food corer 200 may be constructed as any shape for easy handling such as triangular, square, octagonal, circular, oval, and the like.

In some embodiments, the plunger 210 and the other parts of the sampler may be in different colors. As a non-limiting example, the plunger may be in green color and the corer may be transparent. The increased contrast provides a clear view of the position of the plunger with respect to the sampler. In other embodiments, the sampling corer 200 may be further provided with a means for weighing a test sample being picked up, such as a spring, a scale or the equivalent thereof. As a non-limiting example, the sampling corer 200 may be provided with a weigh tension module.

The food corer 200 may be made of plastic materials, including but not limited to, polycarbonate (PC), polystyrene (PS), poly(methyl methacrylate) (PMMA), polyester (PET), polypropylene (PP), high density polyethylene (HDPE), polyvinylchloride (PVC), thermoplastic elastomer (TPE), thermoplastic urethane (TPU), acetal (POM), polytetrafluoroethylene (PTFE), or any polymer, and combinations thereof.

In some embodiments, the sampler may be further configured to be user friendly. For example, the handle 220 may comprise a textured surface to create better visual and tactile differentiation between the grip area and sample areas, communicating the user where to hold the sampler 200.

The sampler (e.g., the corer 200) may be operatively associated with an analytical cartridge (e.g., the disposable cup 300) and/or a detection device (e.g., the device 100). Optionally, the sampler may comprise an interface for connecting to the cartridge. Optionally, a cap may be positioned on the proximal end of the sampler. The sampler 200 may also comprise a sensor positioned with the sampler 200 to detect a presence of a sample in the sampler.

### Disposable Analytical Cartridge

In some embodiments, the present disclosure provides an analytical cartridge or vessel. As used herein, the terms “cartridge”, “vessel”, “pod” and “test cup” are used interchangeably. The analytical cartridge is constructed for implementing a detection test. As used herein, the analytical cartridge is also referred to as an analytic module. The analytical cartridge is disposable and used for one particular target or a particular set of targets to be detected. A disposable analytical cartridge is constructed for processing a test sample to a state permitting the allergen(s) of interest to engage in an interaction with a detection agent, for example, dissociation of samples and protein extraction, filtration of processed particles, storage of reaction solutions/reagents and detection agents, capture of a molecule of interest using detection agents such as antibodies and nucleic acid molecules that specifically bind to targets. In some embodiments, the detection agents may be antibodies specific to target proteins, such as antibodies specific to peanut allergen proteins Ara H1. In other embodiments, the detection agents may be any agents, e.g., nucleic acids such as aptamers, chemical compounds, peptide aptamers and complexes that can specifically recognize target proteins. The present disclosure discusses food allergens as examples of molecules of interest that can be detected with the present assemblies. One skilled in the art would understand, any targets (i.e., molecules of interest) in a sample can be detected.

In accordance with the present disclosure, at least one separate analytical cartridge is provided as part of the assembly. In other embodiments, the analytical cartridge may be constructed for use with any other detection systems.

In some embodiments, a disposable analytical cartridge is intended to be used only for one assay in a sample and therefore may be made of low cost plastic materials, for example, acrylonitrile butadiene styrene (ABS), COC (cyclic olefin copolymer), COP (cyclo-olefin polymer), transparent high density polyethylene (HDPE), polycarbonate (PC), poly(methyl methacrylate) (PMMA), polypropylene (PP), polyvinylchloride (PVC), polystyrene (PS), polyester (PET), or other thermoplastics. Accordingly, a disposable analytical cartridge may be constructed for any particular molecule of interest. In some embodiments, these disposable cartridges may be constructed for one particular allergen only, which may avoid cross contamination with other allergen reactions.

In some embodiments, the disposable cartridge is made of polypropylene (PP), COC (cyclic olefin copolymer), COP (cyclo-olefin polymer), PMMA (poly(methyl methacrylate), or acrylonitrile butadiene styrene (ABS).

In other embodiments, these analytical cartridges may be constructed for detecting two or more different molecules s in a test sample in parallel. In some aspects, the cartridges may be constructed for detecting two, three, four, five, six, seven, or eight different molecules in parallel. In one aspect, the presence of multiple molecules, e.g., two, three, four, five, or more, are detected simultaneously, a positive signal may be generated indicating which molecule is present.

In some embodiments, the disposable analytical cartridge may further be constructed to comprise a bar code that can store the lot specific parameters. The stored information may be later read and stored in any digital formats by the user.

In some embodiments, the analytical cartridge comprises a homogenizer configured to produce a homogenized sample, thereby releasing the molecule of interest from a matrix of the sample into an extraction buffer. The analytical cartridge also comprises a first conduit to transfer the homogenized sample through a filter system to provide a filtrate and a second conduit to transfer the filtrate, making the filtrate to be contacted with detection agents. The first and second conduits comprise a plurality of fluidic paths connecting different parts of the conduits from transferring the processed sample, buffers, filtrate, waste, and other fluids.

In some embodiments, the analytical cartridge may further comprise a rotary valve system providing a mechanism for controlling the transfer of the sample and other fluidic components (e.g., buffer, solution, filtrate, and waste) in the analytical cartridge. The rotary valve switching system may be further configured to provide a closed position to prevent fluid movement in the analytical cartridge.

In some embodiments, the homogenizer and the rotary valve system may be powered by motors located in the detector unit when the analytical cartridge is accepted by the detector unit, or any other motor mechanisms provided by a connected detection device.

In some embodiments, the analytical cartridge is constructed to comprise one or more separate chambers, each configured for separate functions such as sample reception, protein extraction, filtration, storage for buffers, agents and waste solution, and detection reaction. The chambers are separate but connected for operation. In some embodiments, the analytical cartridge may include a sample processing chamber, a detection chamber (also referred to as reaction chamber), a waste chamber, and optionally a buffer chamber. In some embodiments, the analytical cartridge may further comprise a separate filtrate chamber to hold the filtrate and optionally further concentrate the filtrate prior to the transfer to the detection chamber. In some examples, the detection chamber may comprise a detection sensor and an optical window. The detection mechanism of the detector unit analyzes the detection reaction through the optical window to identify the interaction of the molecule of interest with the detection agent in the detection chamber. The detection window is operatively associated with the detection mechanism of a detection device.

In some embodiments, the analytical cartridge comprises a detection sensor for measuring the interaction between the target molecule and the detection agent. The detection sensor is included in the detection chamber. In one non-limiting example, the detection sensor is a transparent substrate which includes a plurality of fluidic channels and a detector chip area. The substrate is referred to as a chipannel, wherein the fluidic channels and the detector chip area are connected. In some examples, the chipannel is a plastic substrate.

In some embodiments, the detector chip area within the chipannel comprises at least one reaction panel and at least one control panel. In other embodiments, the detector chip area within the chipannel may comprise one reaction panel and two control panels. In other embodiments, the chipannel may comprise a plurality of reaction panels and a plurality of control panels. Optionally, the detector chip area of the chipannel further comprises one or more fiducial spots that guide image processing by an imaging mechanism (e.g., a camera) of the detector unit. Any suitable fiducial object may be spotted as a fiducial marker for reference.

In some embodiments, the chipannel comprises antibodies immobilized on the reaction panel of the detector chip area. The detector chip area of the chipannel may further include an optically detectable control probe molecule immobilized on the control panel(s), for normalization of signal output measured by the detection mechanism. In some embodiments, the control probe molecule is an antibody that does not bind to the molecule of interest.

In one preferred embodiment, the chipannel is a plastic chip wherein the reaction panel is printed with antibodies that specifically bind to a molecule of interest that is to be tested and wherein the control panel is printed with control antibodies.

In some embodiments, antibodies, buffers such as extraction buffers and wash buffers, and other components necessary for assembling a functional cartridge are included.

In some embodiments, the analytical cartridge may comprise a data chip unit configured for providing the cartridge information.

In accordance with the present disclosure, the analytical cartridge may be construed in any suitable shape and size. Some exemplary embodiments of the analytical cartridge are illustrated below. The exemplary embodiments do not intent to limit the design of the cartridge.

In some embodiments, homogenization buffer with magnesium chloride (MgCl2) is filled into the filtrate chamber of the analytic cartridge. The concentration of MgCl2 ranges from 10 mM to 100 mM, or from 20 to 80 mM, or from 20 mM to 50 mM.

In some embodiments, the reaction chamber is washed once, twice or three times before reading the reaction signal. The wash buffer may contain magnesium chloride at a concentration from 0.1 mM to 1 mM, such as 0.25 mM.

### Exemplary Embodiments of the Analytical Cartridge

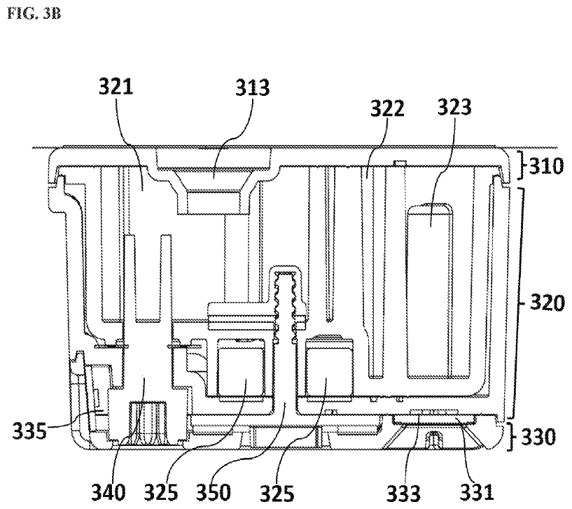

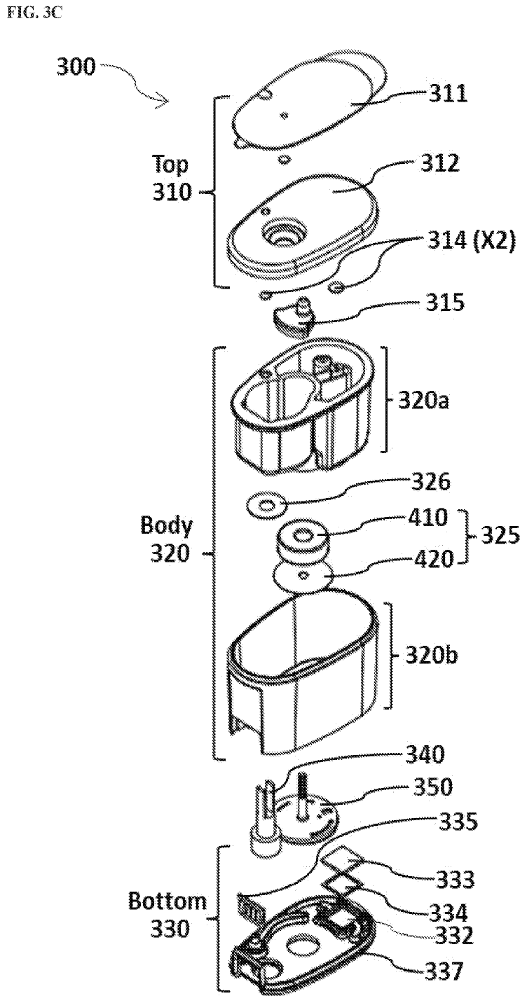

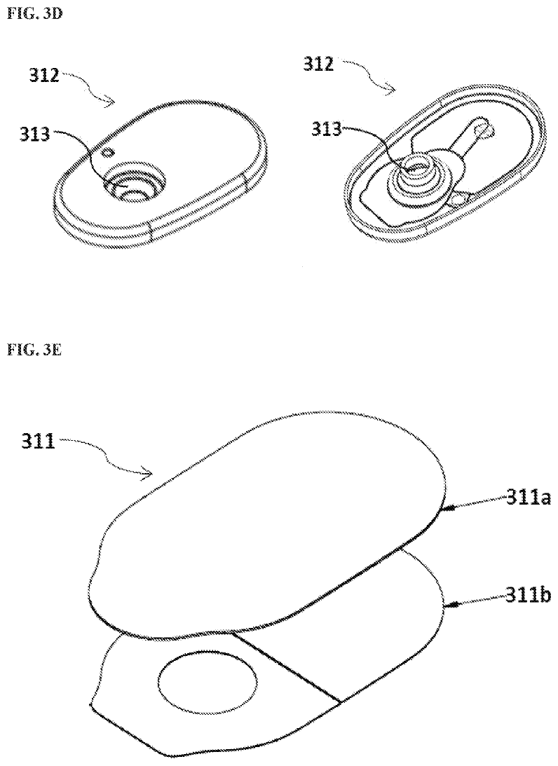

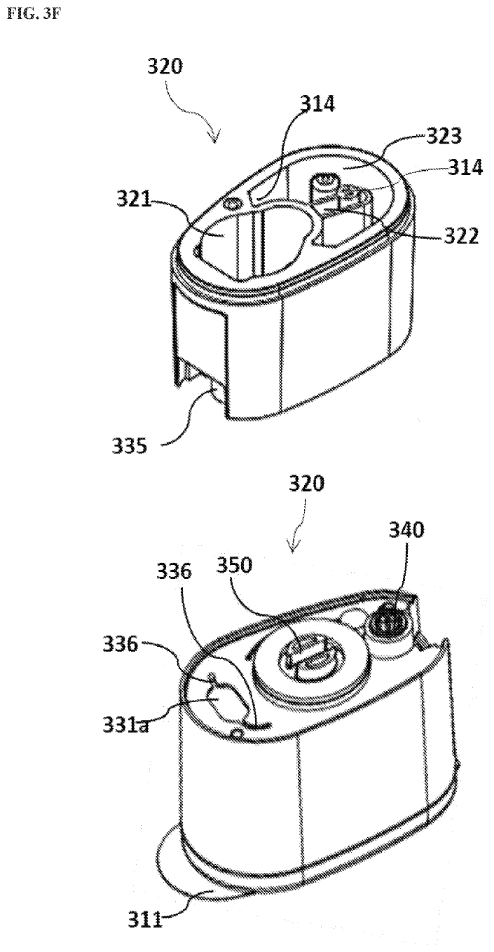

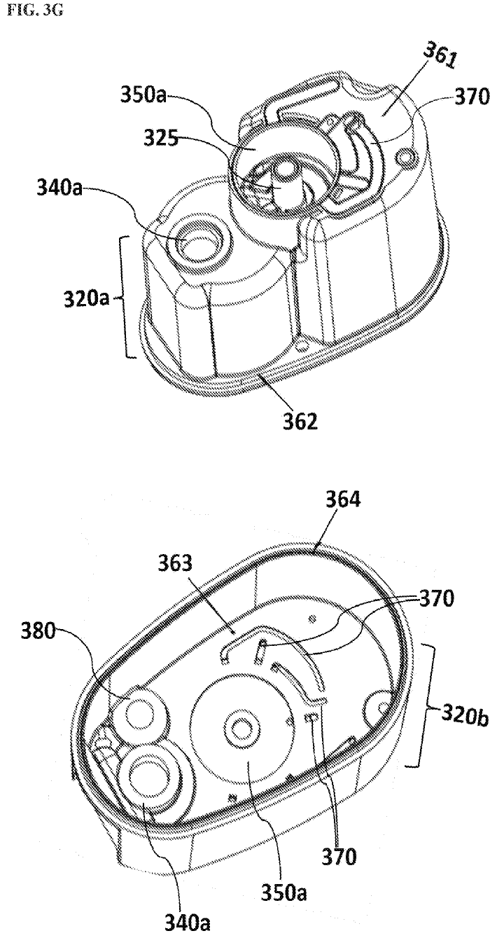

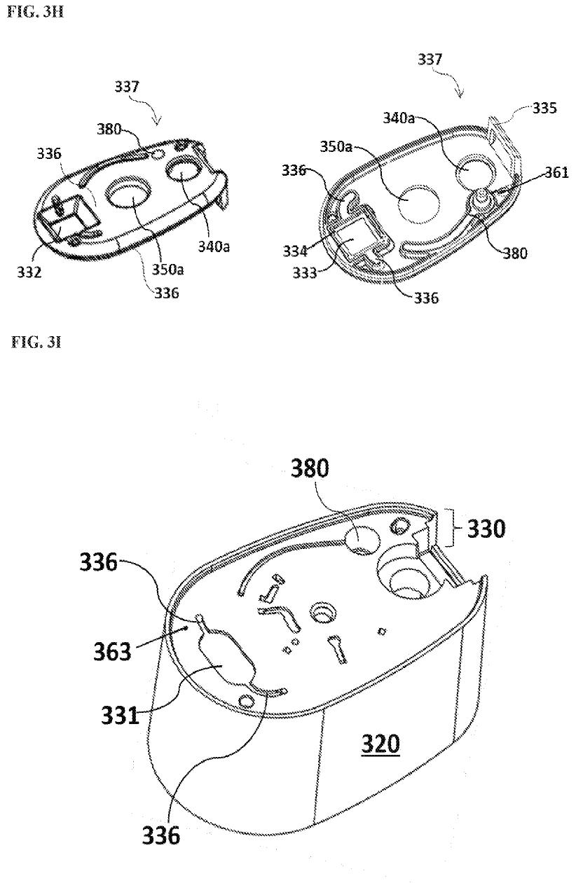

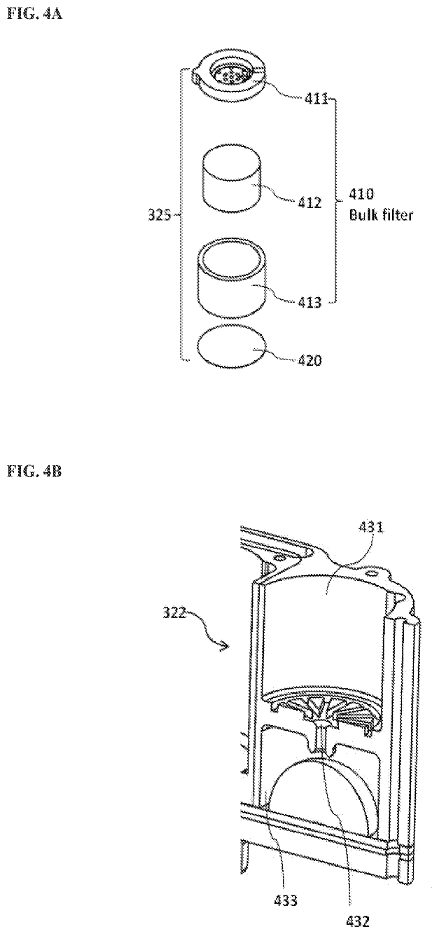

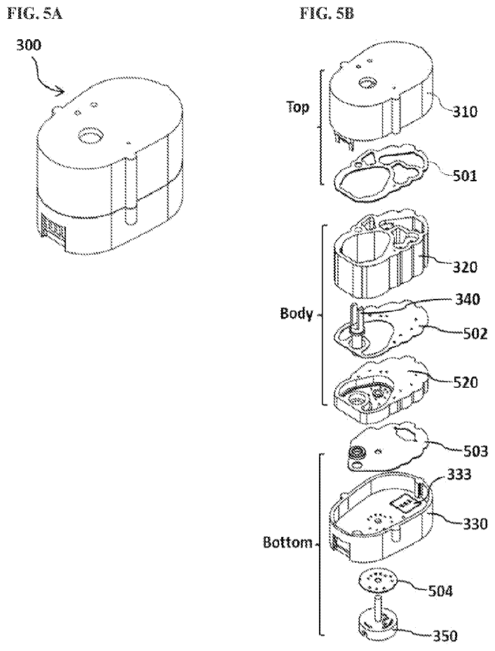

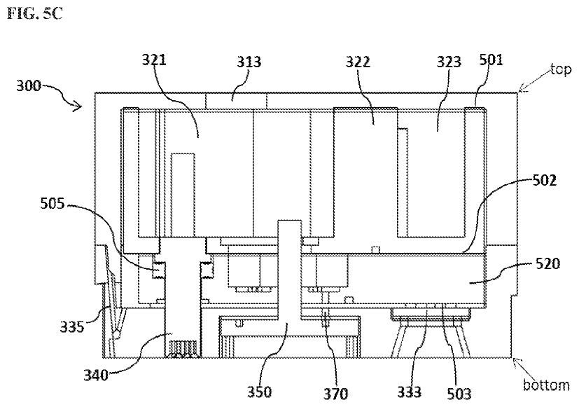

In some embodiments, the disposable analytical cartridge may be construed as a disposable test cup or a cup-like container. The cup container may comprise several compartments that are assembled into a functional analytic module. According to one embodiment of the test cup, as shown in FIG. 3A, the assembled disposable test cup 300 comprises three parts: a cup top 310, a cup body 320 and a cup bottom 330. The three parts are operatively connected to assemble a functional analytical module. The cup 300 further comprises a homogenization rotor 340 that rotates in both directions to homogenize the sample, a filter assembly 325 filtrating the processed sample, a rotary valve 350 contemplated to control the fluid flow inside the cup (FIG. 3B), and fluidic paths transporting the processed sample, mixer, filtrate, buffers and agents to different compartments of the test cup (not shown in FIG. 3B).

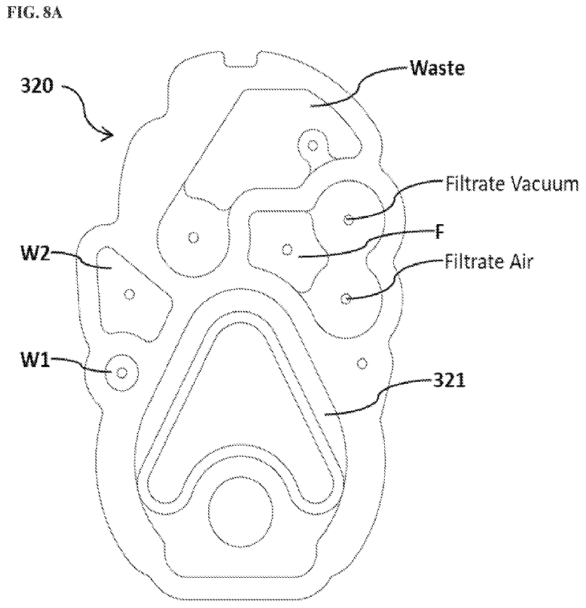

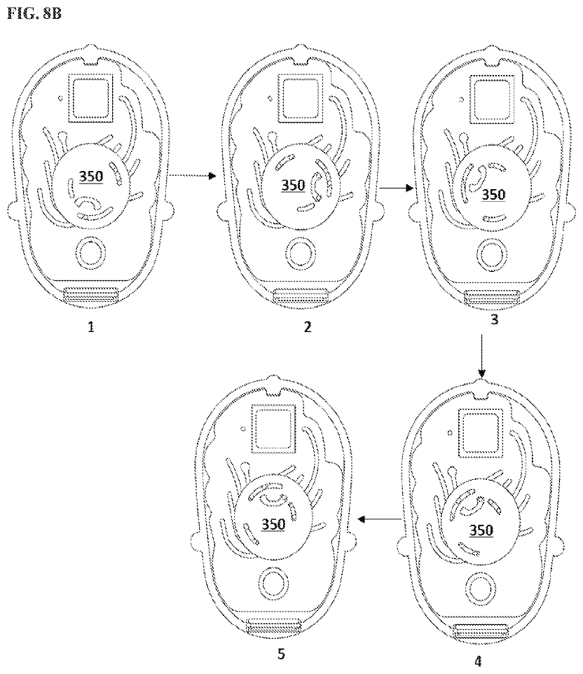

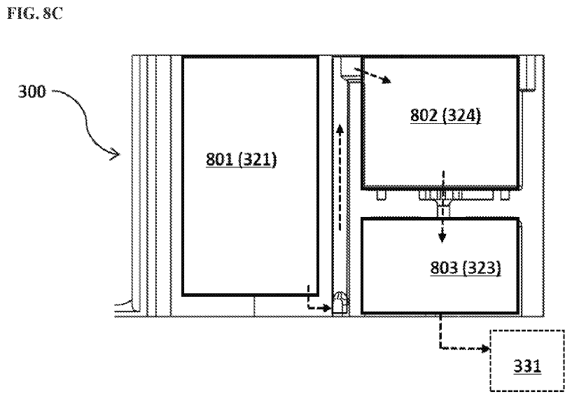

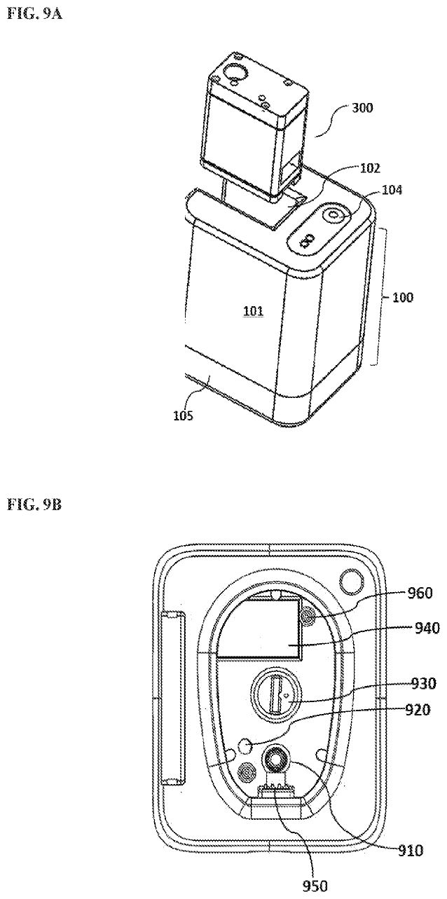

The test cup body 320 may include a plurality of chambers. In one embodiment, as shown in FIG. 3B, the test cup body 320 includes one homogenization chamber 321 comprising a sample processing reservoir 801 (as shown in FIG. 8C), a filtrate chamber 322 for collecting a sample solution after being filtered through the filter (e.g., the 2-state filter 325 shown in FIG. 3B and FIG. 4A), a waste chamber 323 comprising a waste reservoir 803 (as shown in FIG. 8C), and optionally, a wash buffer storage chamber 324 comprising wash buffer storage reservoir 802 (as shown in FIG. 8C). Optionally, one or more separate wash compartments may be included in the cup body 320. In some embodiments, a reaction chamber 331 at the cup bottom 330 for receiving the processed sample (also referred to herein as a signal detection chamber) is included shown in FIGS. 3B and 3H. The reaction/detection chamber 331 may comprise a separate detection sensor (e.g., the chip 333 shown in FIG. 3B) with a detection probe (e.g., antibody) that reacts with the processed sample. All analytical reactions occur in the reaction/detection chamber 331, and a detectable signal (e.g., a fluorescence signal) is generated therein. In some embodiments, detection agents (e.g., antibodies) for example, which are pre-stored in the homogenization chamber 321, may be premixed with the test sample in the homogenization chamber 321, where the test sample is homogenized and the extracted proteins react with the detection agents. The mixed reaction complexes may be transported to the filter 325 before they are transported to the reaction/detection chamber 331. In other examples, detection agents (e.g., antibodies) may be stored in the filtrate chamber 322. The processed sample is filtered through the filter assembly 325 and reacts with the detection agents stored in the filtrate chamber 322. The filtrate containing the molecule of interest and detection agents is transferred to the detection chamber 331 wherein the detection agents engage an interaction with the detection probes immobilized on the sensor (e.g., the chip 333) and the detection signal is measured.

In alternative embodiments, more than one buffer and reagent storage reservoir may be included in the buffer and reagent storage chamber 324. As a non-limiting example, the extraction buffer and wash buffers may be stored separately in a reservoir within the buffer storage chamber 324.

FIG. 3C shows an exploded view of one exemplary embodiment of the disposable test cup 300 which is configured to contain three main components, the top 310, the housing or body 320 and the bottom 330. The cup top 310 may include a cup lid 311, a top cover 312, two or more breather filters 314 which are included to ensure that only air is brought in and that fluids do not escape from the test cup 300. The cup body 320 is composed of two separate parts: an upper housing 320a and an outer housing 320b. The cup bottom assembly 330 includes a bottom cover 337 that sandwiches other components including the reaction chamber 331 (in FIGS. 3F and 3H), a detection sensor, i.e., a chip 333, and a chip gasket 334 that facilitates the attachment of the chip 333 to the bottom of the specialized sensor area 332 in the reaction chamber 331. In some embodiments, the processed sample mixer flows to the reaction chamber 331 and reacts with the detection agents on the chip 333 to generate detectable signals. For example, the chip 333 may be printed with antibodies that specifically bind to the molecule of interest in the test sample. The bottom cover 337 also comprise a port/bit 340a for holding the homogenization rotor 340 and a port/bit 350a for holding the rotary valve 350 (as shown in FIG. 3H). These bits provide a means for linking the homogenization rotor 340 and the rotary valve 350 to the motors of the detection device 100. In some embodiments, a rotor gasket 326 may be configured to the upper housing 320a to seal the rotor 340 to the housing 320, to avoid leakage of fluids. In some embodiments, the bottom cover may further comprise fluidic paths and air channels.

In some embodiments, the cup may further be constructed to comprise a bar code that can store the lot specific parameters. In one example, the bar code may be the data chip 335 that stores the cup 300 specific parameters, including the information of detection agents such as antibodies, expiration date, manufacture information, etc.

FIG. 3D further demonstrates the features of the top cover 312 of the cup shown in FIG. 3A. A corer port 313 is included for receiving a food corer 200, thereby receiving the picked test sample and transferring the sample to the sample processing chamber 321 (also referred to as homogenization chamber). As a non-limiting example, the port 313 may be configured for receiving the sampling corer 200 shown in FIG. 2B. The top cover 312 may also include at least one small hole (FIG. 3D) for air to be drawn in for fluid flow. As a non-limiting example, the top part may have two lids 311. As discussed hereinabove, the lid 311 may comprise two layers: a top lid 311a for sealing and labeling and a bottom 311b for resealing during operation. As shown in FIG. 3E, the second lid at the bottom 311b is constructed for resealing and liquid retention during the operation. The top lid 311a may be peeled back for inserting the test sample collected by the corer 200, and then reclosed after assay completion.

FIG. 3F is a top view of a cup housing body 320 as the upper housing 320a and the outer housing 320b are assembled together (left panel). The upper housing 320a may comprise one or more chambers which are operatively connected. In one embodiment, the homogenization chamber 321, filtration chamber 322 and waste chamber 323 are included in the housing 320a (left panel). Two breath filters 314 are also added to the upper housing 320a. The bottom of the assembled cup body 320 comprises an opening 331a that connects to the reaction/detection chamber 331 with the inlet and outlet 336 for fluid flow (right panel). In this embodiment, the reaction/detection chamber 331 is formed when the bottom cover 337 is assembled together with the body part (see FIG. 3C) The rotor 340 and the rotary valve 350 may be assembled into the cup to form an analytical cartridge (right panel).

FIG. 3G further illustrates the outer interface of the bottom of the upper housing (320a) (upper panel) and the inner interface of the bottom of the outer housing 320b (lower panel). The two energy director faces 361 (face 1) and 362 (face 2) at the outer interface of the upper housing 320a, interact with the two welding mating faces, face 363 (face 1) and 364 (face 2) at the inner interface of the bottom of the outer housing 320b to retain the housing parts 320a and 320b together to form the cup body 320. Fluid paths 370 are also included to flow liquids to the cup bottom 330. The rotor 340 and the rotary valve 350 are assembled into the cup through the rotor port 340a and the rotary valve port 350a, respectively.

FIG. 3H further illustrates the cup bottom cover 337 of the cup bottom 330 of the cup 300 shown in FIG. 3A and FIG. 3C. The reaction/detection chamber 331 comprises a specialized sensor area 332 where a detection sensor, i.e., the chip 333, is positioned through a glass gasket 334. The glass gasket 334 may be included to seal the chip 333 in place to the bottom of the reaction chamber 331 and to prevent fluid leakage. Alternatively, adhesive or ultrasonic bonding can be used to mate the layers together. In some aspects of the present disclosure, the chip 333 may be configured directly at the bottom of the reaction chamber 331 (e.g., the bottom surface of the sensor area 332) as a component of the cup bottom cover 337 and integrated into the cup body as one entity. The entire unit may be of PMMA (poly(methyl methacrylate)) (also referred to as acrylic or acrylic glass). This transparent PMMA acrylic glass may be used as optic window for signal detection.

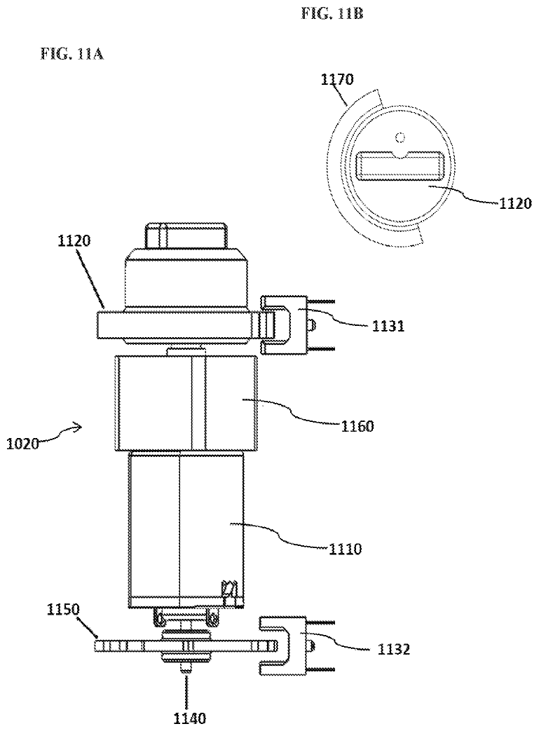

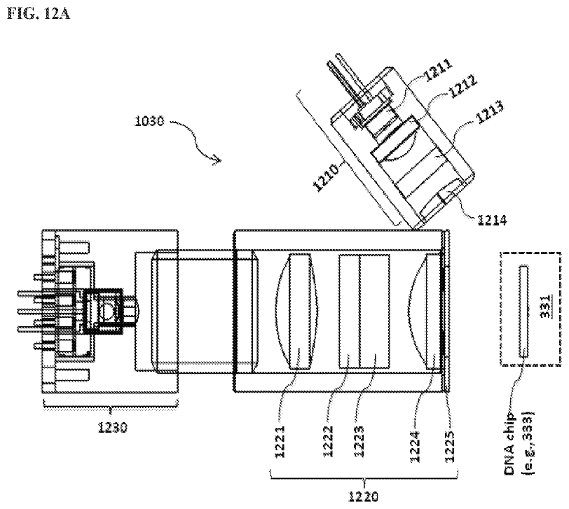

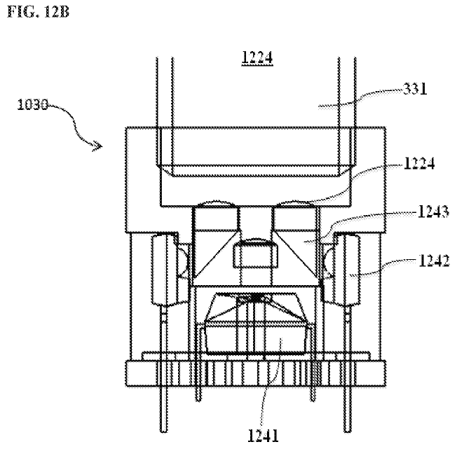

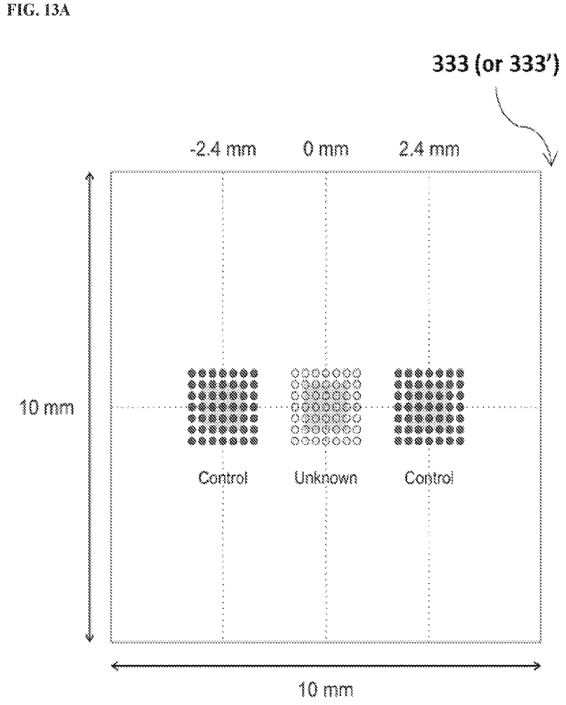

The reaction chamber 331 comprises at least one optical window. In one embodiment, the chamber 331 comprises two optical windows, one primary optical window and one secondary optical window. In some embodiments, the primary optical window serves as the interface of the reaction chamber 331 to the detection device 100, in particular to the optical system 1030 (as shown in FIGS. 10A, 10B, and 12A-12C) of the detection device 100. The detection sensor (e.g., the chip 333) may be positioned between the optical window and the interface of the optical system. The optional secondary optical window may locate at one side of the reaction chamber 331; the secondary optical window allows detection of the background signals. In some aspects of the present disclosure, the secondary optical window may be constructed for measuring scattered light.

As shown in FIG. 3I, the bottom 330 is assembled with the cup body 320. From this bottom perspective view, the bottom surface comprises several interfaces for fluid paths (e.g., fluidic inlet/outlet 336) and a plump interface 380 and the interfaces connecting the rotor 340 and the rotary valve 350 to the detection device 100.

A means may be included to the cup to block the fluid flows between the compartments of the assembled cup 300. In one embodiment, a dump valve 315 (shown in FIG. 3C) in the cup housing 320a is included to block fluid in the homogenization chamber 321 from flowing to the rotary valve 350 that is configured at the bottom of the cup 300. The dump valve 315 is held in place by the rotary valve 350 (FIG. 3C) for shipping, storage, and end of life. The rotary valve 350 locks the dump valve 315 over the filters (e.g., the filter assembly 325) during shipping and prevents fluid flow after completing the detection assay. The rotary valve 350 may be actuated in several steps to direct fluid flow to the proper chambers. As a non-limiting example, the relevant positions of the rotary valve 350 during the detection test are demonstrated in FIG. 8B.

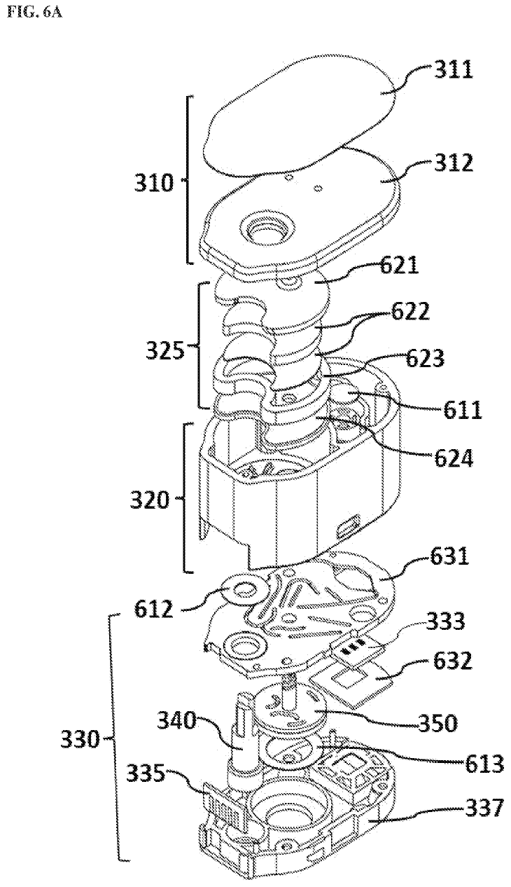

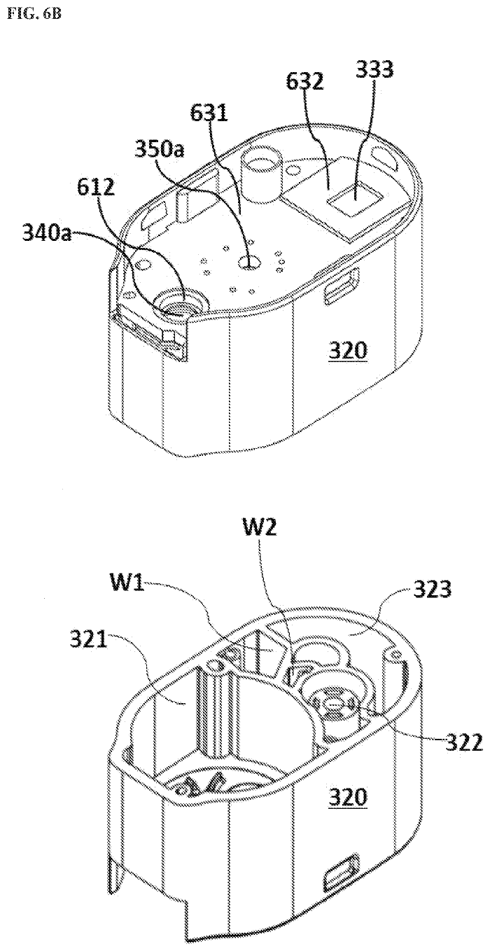

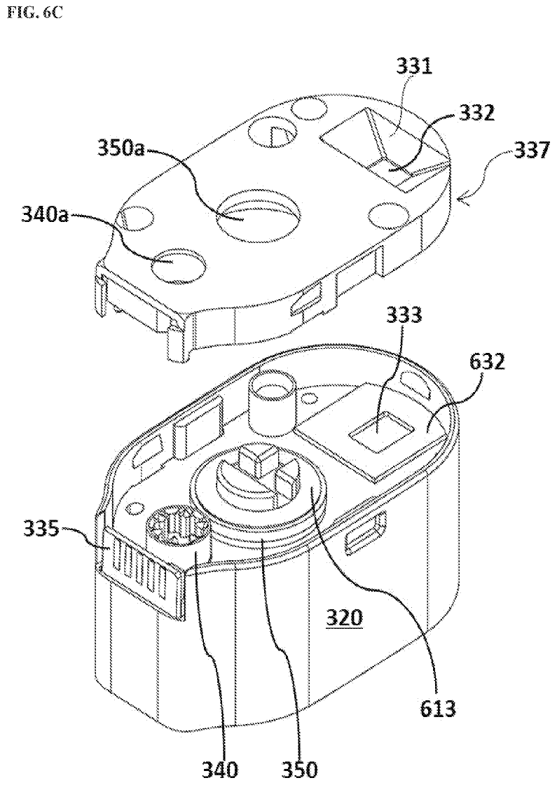

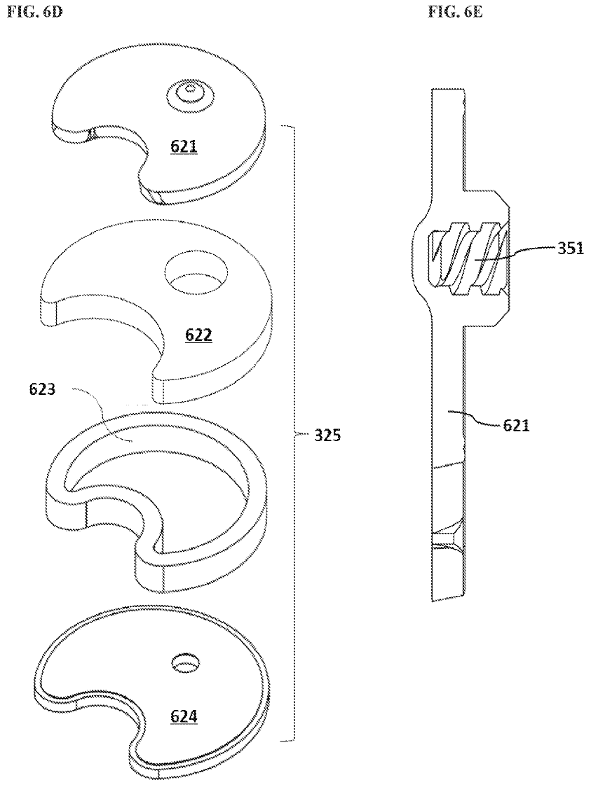

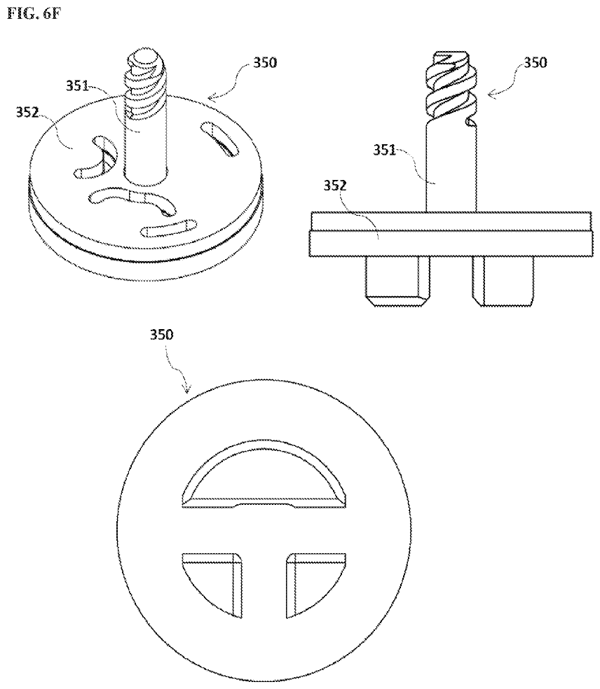

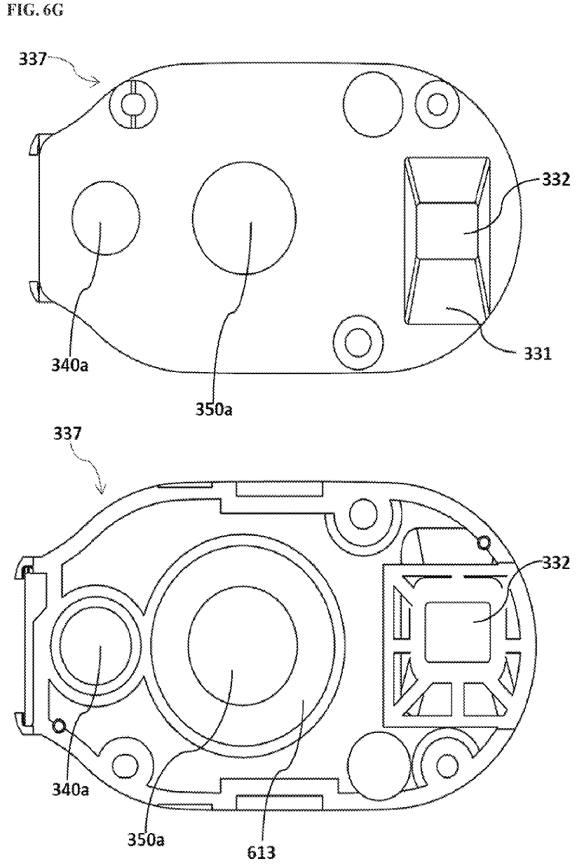

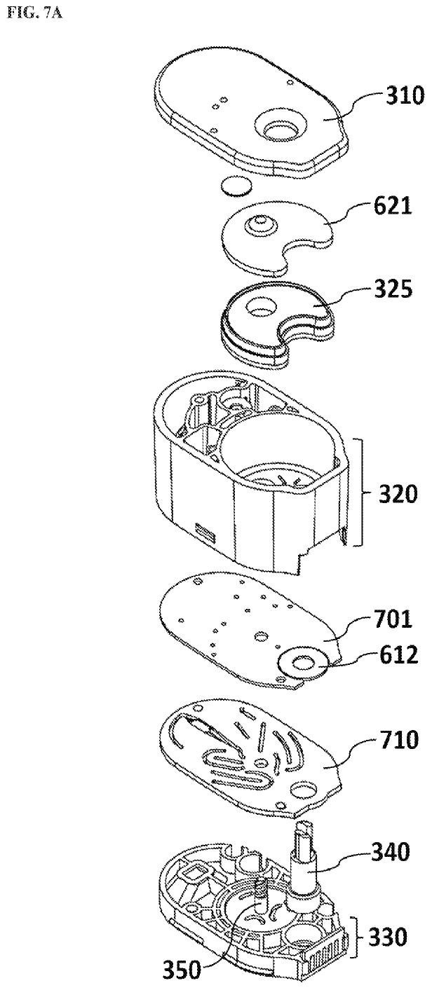

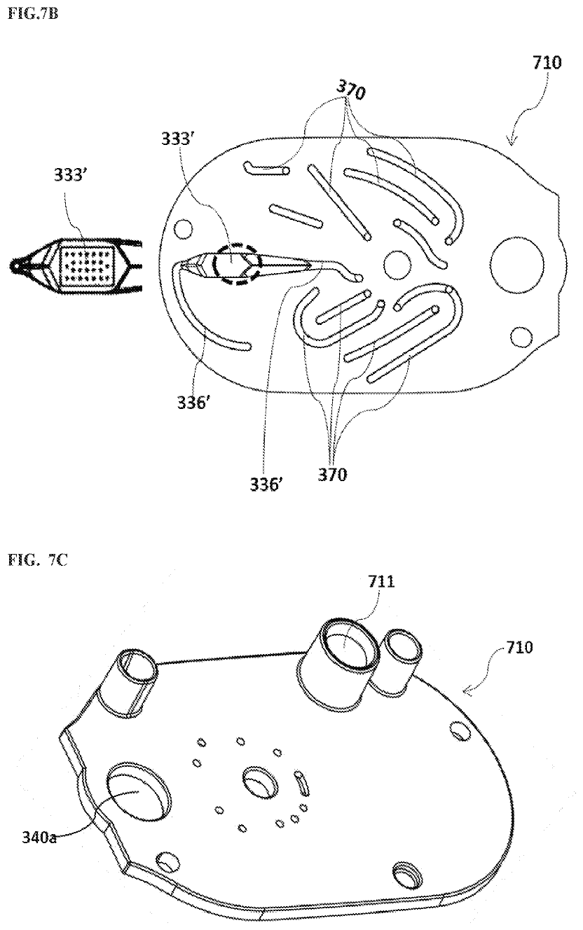

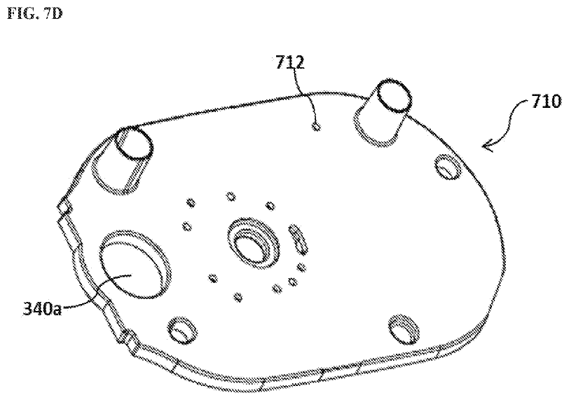

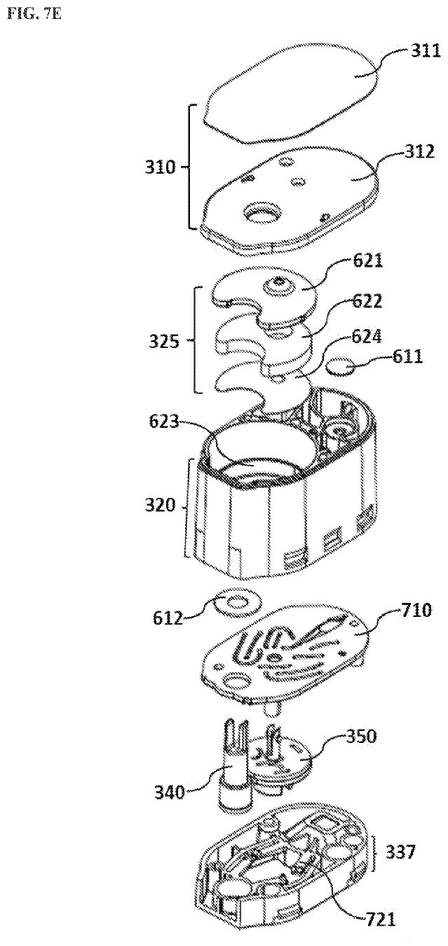

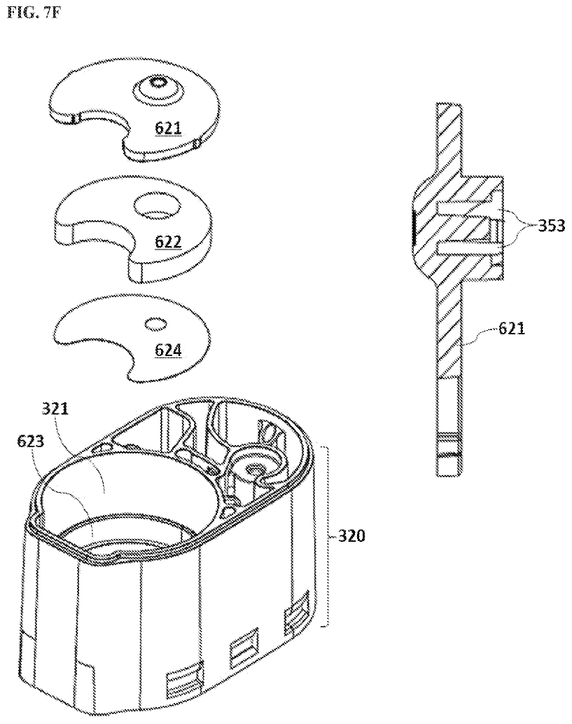

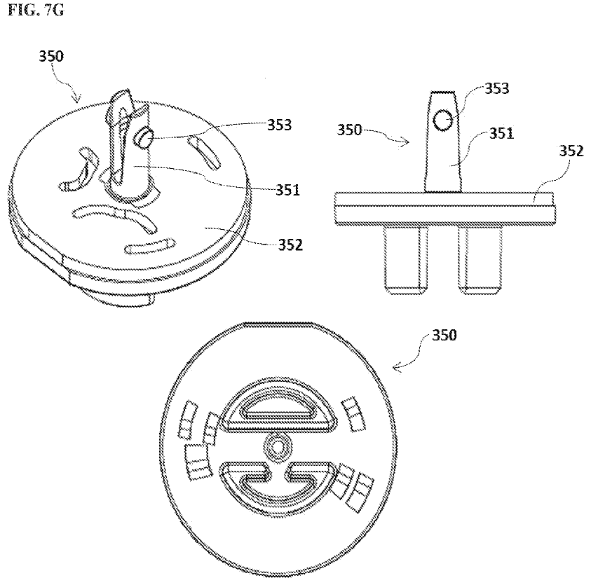

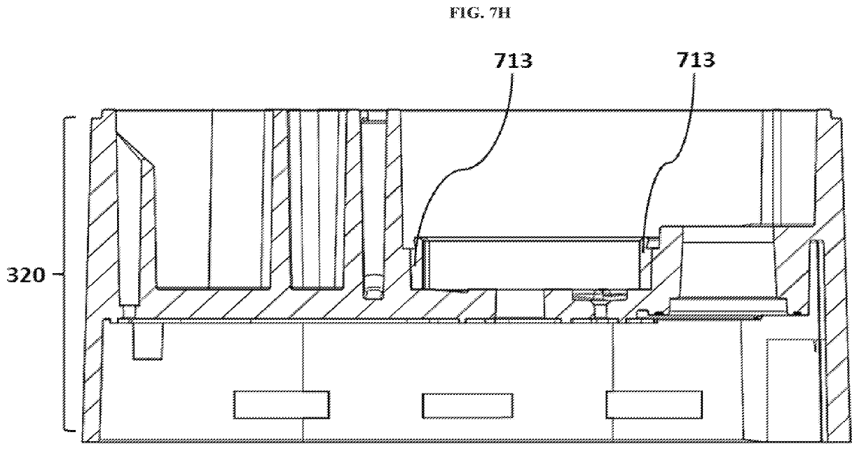

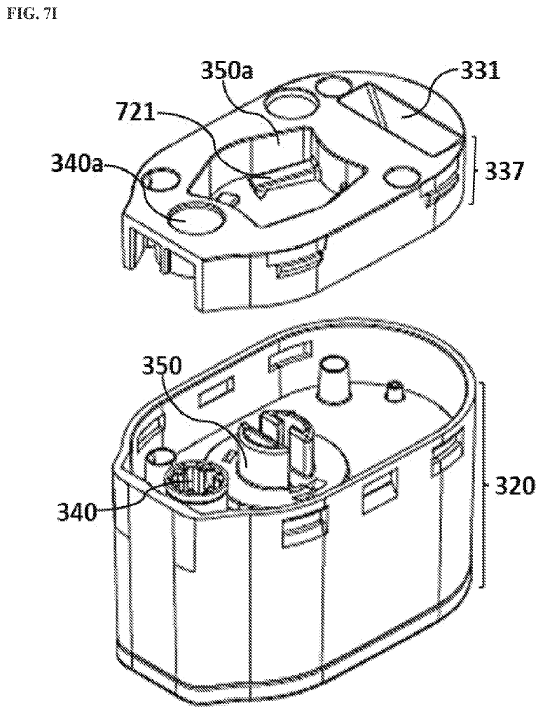

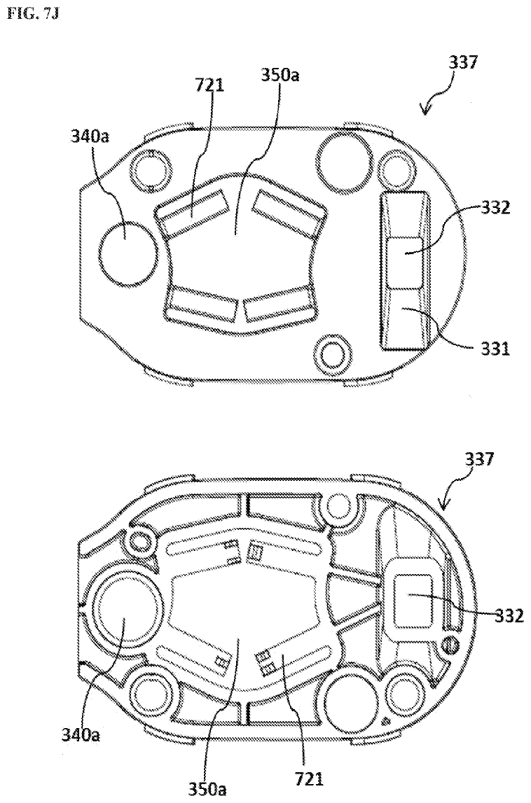

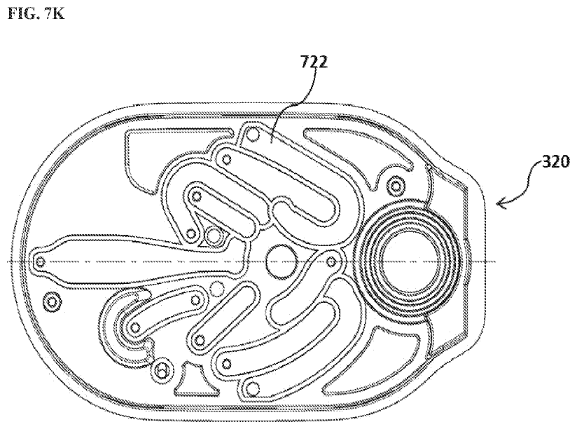

The rotary valve 350 may rotate to regulate the fluid flow through the chambers inside the cartridge. In some embodiments, the rotary valve 350 may comprise a valve shaft 351 that is operatively connected to and locks the dump valve 315 (as shown in FIG. 3C) and a valve disc 352 connected to the valve shaft 351 (e.g., in FIG. 6F). The rotary valve 350 can be attached to the cup through any available means known in the art. In one embodiment, a valve gasket (e.g., the gasket 504 shown in FIG. 5B) may be used. Alternatively, the rotary valve can be attached to the cup through a disc spring (e.g., a wave disc spring). In another embodiment, the rotary valve 350 may be secured to the cup with a plurality of compression coil springs (e.g., 721 shown in FIG. 7J).

In some embodiments, a filter assembly (e.g., the filter 325 shown in FIG. 3C, FIG. 4A and FIG. 6D) is included in the analytical cartridge. The filter removes large particles and other interfering components from the sample, such as fat from a food matrix, before the processed sample is transferred into the reaction chamber 331.

In some embodiments, the filter mechanism may be a filter assembly. The filter assembly may be a simple membrane filter 420. The membrane 420 may be a nylon, PE, PET, PES (poly-ethersulfone), Porex™, glass fiber, or the membrane polymers such as mixed cellulose esters (MCE), cellulose acetate, PTFE, polycarbonate, PCTE (Polycarbonate) or PVDF (polyvinylidene difluoride), or the like. It may be a thin membrane (e.g., 150 μm thick) with high porosity. In some aspects, the pore size of the filter membrane 420 may range from 0.01 μm to 600 μm, or from 0.1 μm to 100 μm, or from 0.1 μm to 50 μm, or from 1 μm to 20 μm, or from 20 μm to 100 μm, or from 20 μm to 300 μm, or 100 μm to 600 μm or any size in between. For example, the pore size may be about 0.02 μm, about 0.05 μm, about 0.1 μm, about 0.2 μm, about 0.5 μm, about 1.0 μm, about 1.5 μm, about 2.0 μm, about 2.5 μm, about 3 μm, about 3.5 μm, about 4.0 μm, about 4.5 μm, about 5.0 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 100 μm, about 150 μm, about 200 μm, about 250 μm, about 300 μm, about 350 μm, about 400 μm, about 450 μm, about 500 μm, about 550 μm, or about 600 μm.

In some alternative embodiments, the filter assembly may be a complex filter assembly 325 (as shown in FIG. 4A) comprising several layers of filter materials. In one example, the filter assembly 325 may comprise a bulk filter 410 composed of a gross filter 411, a depth filter 412, and a membrane filter 420 (FIG. 4A). In one embodiment, the gross filter 411 and the depth filter 412 may be held by a retainer ring 413 to form a bulk filter 410 sitting on the membrane filter 420. In other embodiments, the bulk filter 410 may further comprise a powder that sits inside the filter or on top of the filter. The powder may be selected from cellulose, PVPP, resin, or the like. In some examples, the powder does not bind to nucleic acids and proteins.

In some embodiments, the filter assembly 325 may be optimized for removing oils from highly fatty samples, but not proteins and nucleic acids, resulting in superior sample cleaning. In other embodiments, the ratio of the depth and width of the filter assembly 325 may be optimized to maximize the filtration efficiency.

In some embodiments, the filter assembly 325 may be placed inside a filter bed chamber 431 in the disposable cup body 320. The filter bed chamber 431 may be connected to the homogenization chamber 321. The homogenate can be fed to the filter assembly 325 inside the filter bed chamber 431. The filtrate is collected by the collection gutter 432 (also referred to herein as filtrate chamber) (FIG. 4B). The collected filtrate then can exit the fluidics to flow to the reaction chamber 331 (FIG. 3B). In one example, the collected filtrate may be transported to the reaction chamber 331 from the collection gutter 432 directly. In another example, the filtrate may be first transported to the filtrate collection chamber 433 before being transported to the reaction chamber 331 through the inlet/outlet 336 (FIG. 3H). The fluids may be delivered to the reaction chamber 331 by the fluid paths 370 at the bottom of the cup 320 (as shown in FIG. 3G).

In some embodiments, the filtrate collection chamber 433 may further comprise a filtrate concentrator which is configured to concentrate the sample filtrate before it flows to the reaction chamber 331 for signal detection. The concentrator may be in a half-ball shape, or a conical type concentrator, or a tall pipe.

In accordance, the processed sample (e.g., the homogenate from the chamber 321) is filtered sequentially through the gross filter 411, the depth filter 412 and the membrane filter 420. The gross filter 411 can filter a large particle suspension from the sample, for example, particles larger than 1 mm, and/or some dyes. The depth filter 412 may remove small particle collections and oil components from the sample (such as the food sample). The pore size of the depth filter 412 may range from about 1 μm to about 500 μm, or about 1 μm to about 100 μm, or about 1 μm to about 50 μm, or about 1 μm to about 20 μm, or about 4 μm to about 20 μm, or from about 4 μm to about 15 μm. For example, the pore size of the depth filter 412 may be about 2 μm, or about 3 μm, or about 4 μm, or about 5 μm, or about 6 μm, or about 7 μm, or about 8 μm, or about 9 μm, or about 10 μm, or about 11 μm, or about 12 μm, or about 13 μm, or about 14 μm, or about 15 μm, or about 16 μm, or about 17 μm, or about 18 μm, or about 19 μm, or about 20 μm, or about 25 μm, or about 30 μm, or about 35 μm, or about 40 μm, or about 45 μm, or about 50 μm.

The depth filter 412 may be composed of, for example, cotton including, but not limited to raw cotton and bleached cotton, polyester mesh (monofilament polyester fiber) and sand (silica). In some embodiments, the filter material may be hydrophobic, hydrophilic or oleophobic. In some examples, the material does not bind to nucleic acids and proteins. In one embodiment, the depth filter is a cotton depth filter. The cotton depth filter may vary in sizes. For example, the cotton depth filter may have a ratio of width and height ranging from about 1:5 to about 1:20. The cotton depth filter 412 may be configured to correlate total filter volume and the food mass being filtered.

The membrane filter 420 can remove small particles less than 10 μm in size, or less than 5 μm in size, or less than 1 μm in size. The pore size of the membrane may range from about 0.001 μm to about 20 μm, or from 0.01 μm to about 10 μm. Preferably the pore size of the filter membrane may be about 0.001 μm, or about 0.01, or about 0.015 μm, or about 0.02 μm, or about 0.025 μm, or about 0.03 μm, or about 0.035 μm, or about 0.04 μm, or about 0.045 μm, or about 0.05 μm, or about 0.055 μm, or about 0.06 μm, or about 0.065 μm, or about 0.07 μm, or about 0.075 μm, or about 0.08 μm, or about 0.085 μm, or about 0.09 μm, or about 0.095 μm, or about 0.1 μm, or about 0.15 μm, or about 0.2 μm, or about 0.2 μm, or about 0.25 μm, or about 0.3 μm, or about 0.35 μm, or about 0.4 μm, or about 0.45 μm, or about 0.5 μm, or about 0.55 μm, or about 0.6 μm, or about 0.65 μm, or about 0.7 μm, or about 0.75 μm, or about 0.8 μm, or about 0.85 μm, or about 0.9 μm, or about 1.0 μm, or about 1.5 μm, or about 2.0 μm, or about 3.0 μm, or about 3.5 μm, or about 4.0 μm, or about 4.5 μm, or about 5.0 μm, or about 6.0 μm, or about 7.0 μm, or about 8.0 μm, or about 9.0 μm, or about 10 μm. As discussed above, the membrane may be a nylon membrane, PE, PET, a PES (poly-ethersulfone) membrane, a glass fiber membrane, a polymer membrane such as mixed cellulose esters (MCE) membrane, cellulose acetate membrane, cellulose nitrate membrane, PTFE membrane, polycarbonate membrane, Track-Etched polycarbonate membrane, PCTE (Polycarbonate) membrane, polypropylene membrane, PVDF (polyvinylidene difluoride) membrane, or nylon and polyamide membrane.

In one embodiment, the membrane filter is a PET membrane filter with 1 μm pore size. The small pore size can prevent particles larger than 1 μm to pass into the reaction chamber. In another embodiment, the filter assembly may comprise a cotton filter combined with a PET mesh having 1 μm pore size.

In other embodiments, the filter components may be assembled together by any known methods in the art, such as by heat welding, ultrasonic welding or a similar process that ensures the assembled materials can be die-cut and packaged without damaging or inhibiting the performance of each filter independently or as an integrated filter assembly. In other embodiments, the packaging of each part the filter assembly enables high-speed automation systems on a manufacturing assembly line (e.g., a robotic assembly line).

In some embodiments, the filtration mechanism has low protein binding, low or no nucleic acid binding. The filter may act as a bulk filter to remove fat and emulsifiers and large particles, resulting in a filtrate with comparable viscosity to the buffer.

In some embodiments, the filter assembly 325 including the gross filter 411, the depth filter 412 and the membrane filter 420 can allow the maximal recovery of testing materials, and detection agents.

In other embodiments, the filtration assembly 325 may be configured to comprise a filter 624 (e. g., a mesh filter) that is inserted to a filter gasket 623, a bulk filter 622 composed of a gross filter and a depth filter and a filter cap 621 (as shown in FIG. 6D). In an alternative embodiment, the filter gasket 623 can be molded into the cup body as an overmolded component of the cup body 320. e.g., in the homogenization chamber 321 (as shown in FIGS. 7E and 7F). The filter 624, the bulk filter 622 and the filter cap 621 are inserted to the overmolded gasket to form a functional filter assembly 325.

In some embodiments, the filtration mechanism can complete the filtering process in less than 1 minute, preferably in about 30 seconds. In one example, the filtration mechanism may be able to collect the sample within 35 seconds, or 30 seconds, or 25 seconds, or 20 seconds with less than 10 psi pressure. In some embodiments, the pressure may be less than 9 psi, or less than 8 psi, or less than 7 psi, or less than 6 psi, or less than 5 psi.

In some alternative embodiments, the filtration chamber 322 may comprise one or more additional chambers conjured for filtering the processed sample. As illustrated in FIG. 4B, the filtration chamber 322 may further comprise a separate filter bed chamber 431 wherein a filter assembly 325 (as illustrated in FIG. 4A) is inserted and connected to a collection gutter 432. The collection gutter 432 is configured to collect the filtrate that runs through the filter assembly 325, and the gutter 432 may be directly connected to the flow cell fluidics to flow the filtrate to the reaction chamber 331 for signal detection. Optionally, another collection/concentration chamber 433 may be included in the filtration chamber 322 which is configured for collecting and/or concentrating the filtrate collected through the collection gutter 432 before the filtrate is transported to the reaction chamber 331 for signal detection. The collection/concentration chamber 433 is collected to the filter bed chamber 431 through the collection gutter 432.

FIGS. 5A to 5C illustrate another embodiment of the analytical cartridge. FIG. 5A illustrates an alternative assembly of the test cup 300. The components of the cup 300 of this embodiment are shown in FIG. 5B. According to this embodiment, the cup 300 comprises three parts, a cup top including a cup top cover 310, a cup body comprising a cup tank 320, and a cup bottom including a cup bottom cover 330, which are operatively connected to form an analytic module. As illustrated in FIG. 5B, the top of the cup is a top cover 310 for sealing the cup where a test sample is placed into the cup for testing. A top gasket 501 may be included to seal the top 310 to the cup body 320. The upper cup body 320 comprises the homogenization chamber, waste chamber, chambers for wash buffers (e.g., wash 1 chamber (W1), wash 2 chamber (W2) (shown in FIG. 6B, right panel), and air vent stacks for controlling air and thus fluid flow. A rotor 340 is configured in the homogenization chamber for homogenizing the test sample in an extraction buffer. The shape of the rotor may be adjusted to fit the cup during the assembly. A mid gasket 502 is located at the bottom of the upper cup body 320 to seal the body 320 to the manifold 520 with holes for fluid flow. The manifold 520 is configured to hold the filter 325 and the fluid paths 370 for fluid flow. Another mid gasket 503 is added to seal the manifold 520 to the cup bottom 330, where the reaction chamber (e.g., chamber 331), the detection sensor (e.g., glass chip 333), glass gasket (e.g., gasket 334) and the memory chip (e.g., EPROM) are located. The rotor 340 is sealed to the bottom through an O-ring 505 (shown in FIG. 5C). The rotary valve 350 is configured to the cup 300 at the bottom 330 through a valve gasket 504. In another embodiment, the rotary valve 350 can be configured to the cup 300 through a spring arm, such as wave disc springs and compression coil springs at the cup bottom 330 (e.g., 721 shown in FIG. 7J). The configuration of each components of the cup in FIG. 5A is also illustrated in a section view in FIG. 5C.

According to the present disclosure, a third embodiment of the disposable cup 300 is illustrated in FIG. 6A. FIGS. 6B-6G further illustrate the components of the disposable cup 300 in FIG. 6A. In this embodiment, the configurations of the detection sensor (i.e., the chip printed with detection agents such as SPNs or antibodies) and fluidic paths are further integrated. As shown in FIG. 6A, the cartridge comprises a top part 310, a body part 320 and a bottom part 330. The rotor 340 is sealed to the cup body 320 through a gasket 612. The rotary valve 350 is assembled to the cartridge through a disc spring 613, or alternatively through compression coil springs at the cup bottom part 330 (e.g., 721 shown in FIG. 7J). When implementing a detection assay, the rotary valve 350 may rotate and move the seal 612 to free the rotor 340 for homogenizing the test sample. In this embodiment, a separate panel 631 is provided between the bottom of the cup body 320 and the bottom cover 337 in which the fluidic channels are included. This separate panel 631 with fluidic channels functions equivalently as the fluidic paths 370 of the previous cup embodiments (e.g., FIGS. 3C, 3G and 3I). The sensor chip 333 may be operatively connected to the fluidic panel 631 and the sensor area 332 of the reaction chamber 331 in the bottom cover 337 through a chip PSA 632. In an alternative embodiment, the sensor chip 333 and the fluidic panel 631 may be combined to form a single thin panel (also referred to as a chipannel), therefore forming a separate chipannel 710 (as shown in FIG. 7A). The chipannel 710 is discussed in detail below.

The cup top 310 may comprise a top lid 311 having two labels 311a and 311b as shown in FIG. 3E, and a top cover 312 as shown in FIG. 3D. The cup body 320 may be configured for comprising several separate chambers, including a homogenization chamber 321, a filtration chamber 322, a waste chamber 323, two or more washing spaces (W1 and W2) as shown in FIG. 6B (right panel). In some examples, the filtration chamber 322 has a vent 611 (shown in FIG. 6A). The wetting of the vent 611 can signal to the pressure sensor of the electronics that the chamber 322 is full (FIG. 6B). Similar to other designs, at the bottom of the cup body 320 (FIG. 6B, left panel), several ports are designed including a port 340a for the rotor 340 and a port 350a for the rotary valve 350 (e.g., the rotary valve 350 shown in FIG. 6F) for assembling a functional cartridge. When the cup bottom cover 337 is sealed to the cup body 320 and seals the cup to form an analytic module, these ports are aligned with the ports of the bottom cover 337 (e.g., 340a and 350a as shown in FIG. 6C). The sensor chip 333 is attached to the bottom of the cup body 320 through the chip PSA 632 (FIG. 6B, left panel).

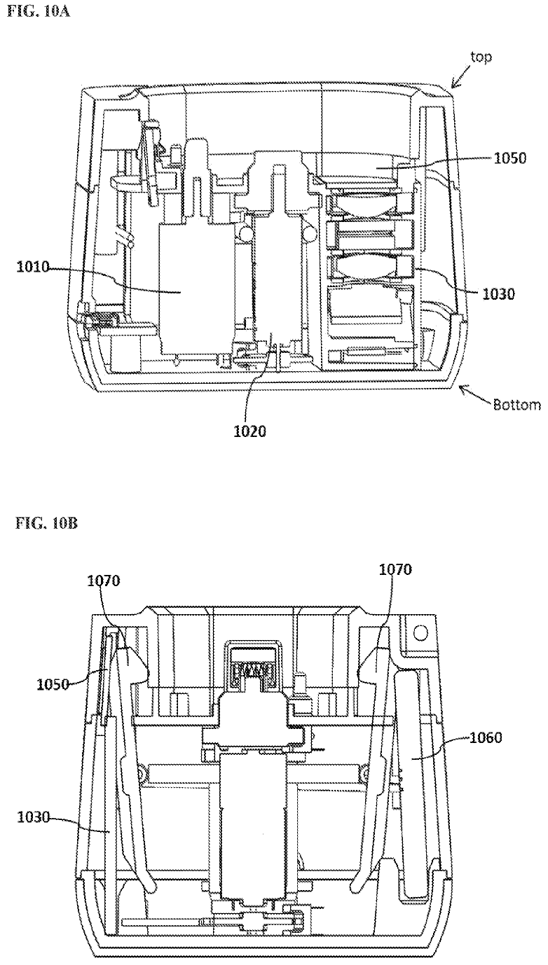

FIG. 6C shows a bottom perspective view of the cup bottom cover 337 and the bottom of the cup body 320 in alignment with each other, indicating the position of each component upon assembly of the test cup. When the bottom cover 337 and the cup body 320 are assembled together, a detection chamber with an optical window (331) is formed wherein a sensor area 332 holds the sensor chip 333. The optical window of the detection chamber 331 provides a connection to the detector unit (e.g., the detection device 100 in FIGS. 1 and 9A).

In this embodiment, the fluidic panel 631 is positioned between the bottom of the cup body 320 and the bottom cover 337 (FIG. 6A); the fluidic panel 631 may be operatively connected to a detection sensor (333). As a non-limiting example, the fluidic panel 631 is connected to the sensor chip 333 through the chip PSA 632 and provides essential fluid paths (e.g., 370) for flowing the processed sample to the detection chamber 331, thereby to the sensor chip 333.

In some examples, a filter assembly 325 is inserted to the homogenization chamber 321 to filtrate the processed sample. In one example, the filter assembly 325 may be the filter illustrated in FIG. 4A. In another example, the filter assembly 325 may be configured to comprise a filter 624 (e. g., a mesh filter) that is inserted to a filter gasket 623, a bulk filter 622 and a filter cap 621 (FIG. 6D). The filter assembly 325 may be fastened and controlled by the rotary valve 350 (FIG. 6E). In this embodiment, the filter cap 621 is engaged in an interaction with the threaded top of the rotary valve shaft 351 (FIG. 6E). The rotary valve 350 comprises a valve shaft 351 that is operatively connected to and locks the filter cap 621, a valve disc 352 connected to the valve shaft 351 (e.g., in FIG. 6F). The valve disc 352 is connected to a motor of the detector unit upon assembling the test cup to the detector unit.

FIG. 6G shows a bottom perspective view (upper panel) and a top perspective view (lower panel) of the cup bottom cover 337. The exterior of the bottom cover 337 holds ports (e.g., 340a and 350a) and the optical window of the sensor area 332 for connecting to the detection device 100. The interior of the bottom cover 337 includes the disc spring 613 to secure the rotary valve 350.

In some embodiments, the reaction chamber 331 at the cup bottom cover 337 may comprise a specialized sensor area 332 which is configured for holding a detection sensor for signal detection. In some aspects of the disclosure, the detection sensor may be a solid substrate (e.g., a glass surface, a chip, and a microwell) of which the surface is coated with detection probes such as short nucleic acid sequences complementary to the SPNs that bind to the target allergen. In some examples, the detection sensor held at the sensing area 332 within the reaction chamber 331 may be a chip 333 (as shown in FIGS. 3C and 6A).

In other embodiments, the reaction chamber 331 comprises at least one optical window. In one embodiment, the chamber comprises two optical windows, one primary optical window and one secondary optical window. Similar to the other embodiments, the primary optical window serves as the interface of the reaction chamber 331 to the detection device 100, in particular to the optical system 1030 (as shown in FIGS. 10A, 10B, and 12A-12C) of the detection device 100. The detection sensor (e.g., the chip 333, and the detection area 333′ of the chipannel 710) may be positioned between the optical window and the interface of the optical system. The optional secondary optical window may locate at one side of the reaction chamber 331; the secondary optical window allows detection of the background signals. In some aspects of the present disclosure, the secondary optical window may be constructed for measuring scattered light.

In some embodiments, the chip 333 and/or the detection area 333′ of a chipannel 710 that is printed with antibodies or nucleic acid molecules, is aligned with the optical window. In some embodiments, the chip comprises at least one reaction panel and at least one control panel. In some aspects, the reaction panel of the chip faces the reaction chamber 331, which is flanked by an inlet and outlet channel 336 of the cartridge 300 (e.g., shown in FIGS. 3H and 3I). In some embodiments, the reaction panel of the chip 333 may be coated/printed with antibodies that specifically bind to the molecules to be detected. s.

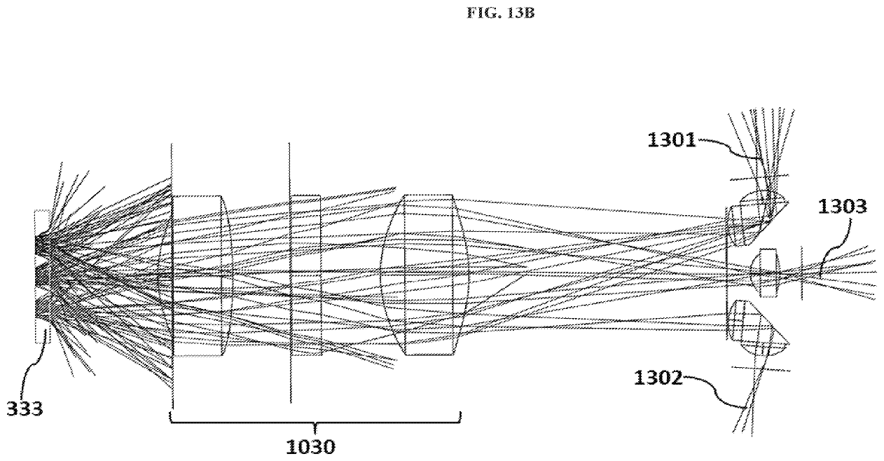

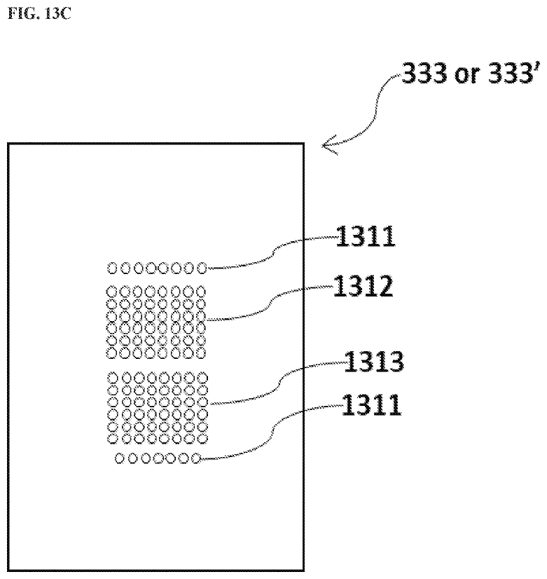

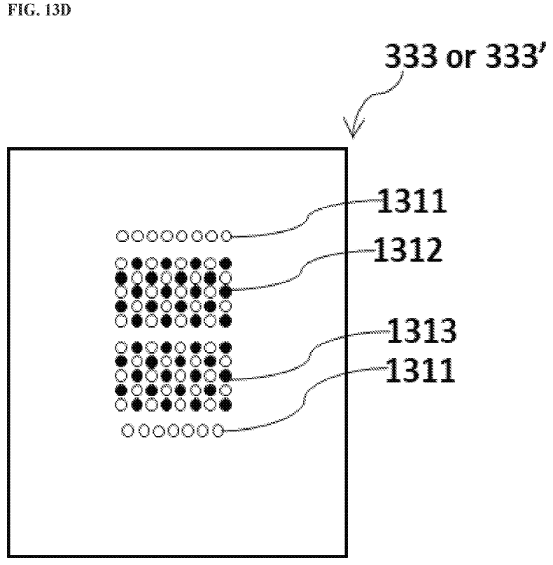

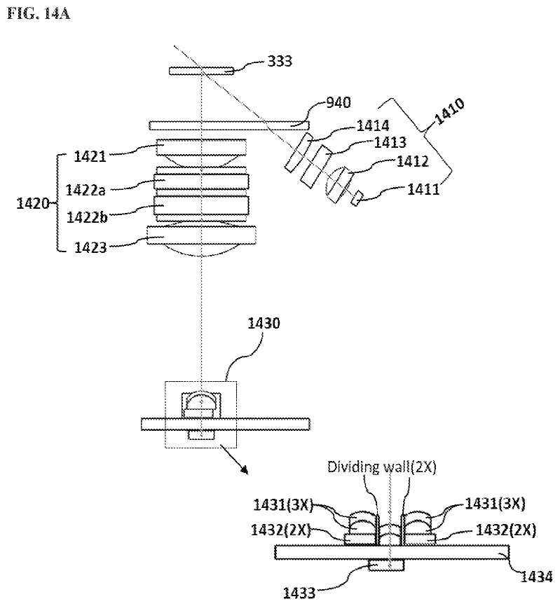

In one preferred embodiment, the sensor chip (e.g., 333 in FIG. 3C, FIG. 5B and FIG. 6A, and 333′ in FIG. 7B) may comprise a reaction panel printed with detection agents, and two or more control areas (control panels) printed with control probes. The reaction panel may be printed with nucleic acid detection probes (e.g., SPNs) or antibodies. In some aspects, the control panels provide an optical set-up with a mechanism to normalize signal output with respect to the reaction panel and to confirm functioning operational procedures. An exemplary configuration of the chip 333 or the detection area 333′ is illustrated in FIG. 13A.

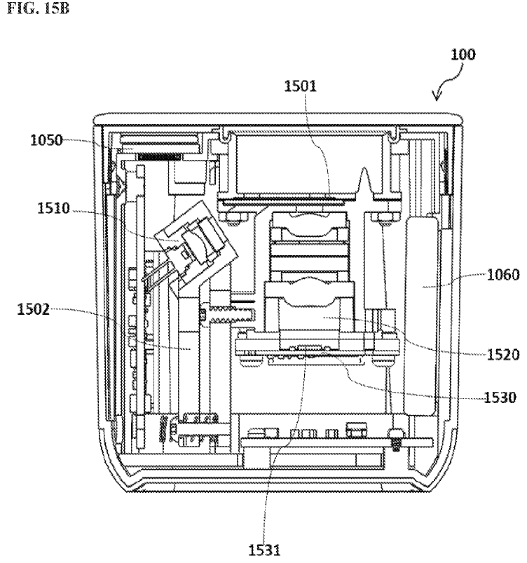

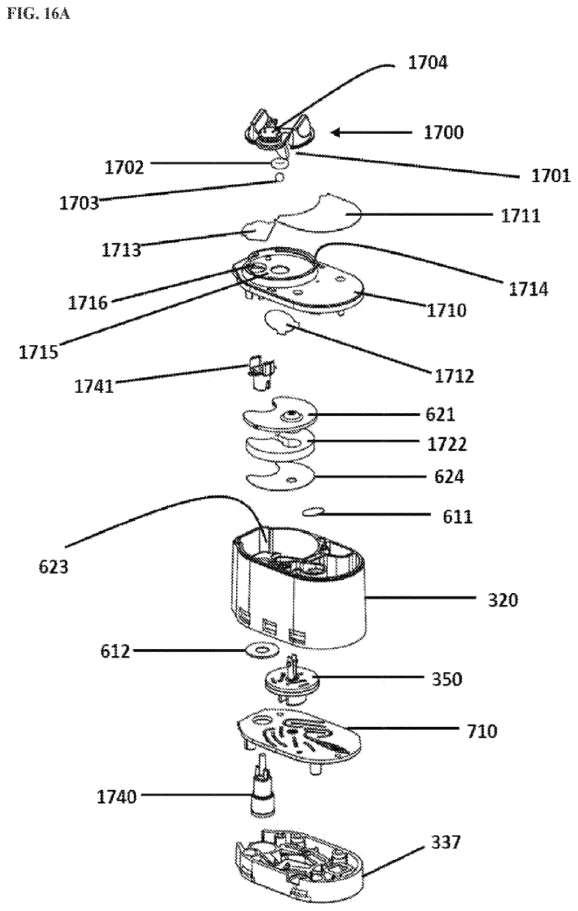

In another embodiment, the sensor DNA chip (e.g., 333 in FIG. 3C, FIG. 5B and FIG. 6A, and 333′ in FIG. 7B) may comprise one reaction panel printed with detection agents such as nucleic acid probes and antibodies, one control area (control panel) and one or more fiducial spots that can guide image processing and provide a self-correction mechanism for an image detector (e.g., a camera detector in FIG. 15A). An exemplary configuration of the chip 333 or the detection area 333′ is illustrated in FIG. 13C.

In some embodiments, the sensor chip may be pre-packed into the reaction chamber 331 of the cartridge, e.g., at the sensing area 332. In other embodiments, the sensor chip may be packed separately with the disposable cartridge (e.g., the cup 300 in FIG. 1). In other embodiments, the chip 333 may be attached to the fluidic panel 631 shown in FIG. 6A. In other embodiments, the chip may be integrated to the chipannel as a specialized detection area of the chipannel (e.g., 333′ of the chipannel 710 shown in FIG. 7B).

Another alternative embodiment of the analytical cartridge is provided in the present disclosure. The configuration of the test cup of this alternative embodiment is shown in FIG. 7A, in which the test cup 300 comprises a similar configuration of the compartments (e.g., shown in FIG. 6A) including a cup top 310, a cup body 320 that is configured to include a homogenization chamber, a filtrate chamber, wash chambers and a waste chamber, and a cup bottom 330. This design is simple and requires fewer components. In this embodiment, a chipannel 710 that combines the fluidic panel 631, the chip 333 and the chip PSA 632 into a single thin piece is provided to replace these components. The chipannel 710 may be connected to the cup body 320 through a gasket 701 (FIG. 7A) and the bottom cover 337 via a port connection 711 (FIG. 7C). Alternatively, the chipannel 710 may be welded to the cup body by a seal face 712 (e.g., in the alternative embodiment shown in FIG. 7D).

In some embodiments, the chipannel 710 comprises the fluidic paths and the sensor chip with detection probes immobilized thereon, which is made of a separate thin plastic polymer. According to the present disclosure, the chipannel 710 may be a piece of plastics in which a specific area (FIG. 7B) is configurated as the detection area 333′ (i.e., an equivalent of the separate sensor chip 333 in other embodiments). The chipannel 710 may comprise the fluidic channels (e.g., the paths 370 in FIG. 7B) connected to the detection area 333′. The detection area 333′ is printed with detection agents and control probes in a specific pattern (FIG. 7B). The detection area 333′ may be flanked by an inlet and outlet channel 336′ (FIG. 7B). The chipannel 710 may be made of optically clear resin such as COC, COP and PMMA.

In some embodiments, antibodies are printed on the detection area 333′ of the chipannel 710 by UV irradiation. In some examples, the detection area 333′ further comprises control probes immobilized thereon. The detection antibodies and control probes are immobilized to form separate reaction panels and control panels. In some embodiments, the antibodies and control probes are printed on the detection area 333′ of the chipannel 710 as shown in FIG. 13C. The detection antibodies and control probes are printed to the reaction panel 1312 and the control panel 1313, respectively. Within each panel, the detection antibodies and control probes are printed in a checkerboard pattern, such as the pattern shown in FIG. 13D. Multiple antibodies may be printed on the chip surface in duplicates or in triplicates. In some embodiments, the secondary antibodies and other non-probe antibodies may be printed as control probes.

FIGS. 7C and 7D illustrate perspective views of the chipannel 710. In one embodiment, the chipannel 710 is held by a port connection 711 (FIG. 7C). A vacuum, for example, the vacuum of the detection device 100 is connected to the chipannel 710 through the port connection 711. In another embodiment, the chipannel 710 is sealed to the cup bottom 337 via a face seal 712 (FIG. 7D). The overmolding of the chipannel 710 and the cup bottom 330 will result in a seamless combination of the parts. Any overmolding and casting techniques, e.g., an injection molding process, may be used to overmold the parts into a single part.

In some embodiments, the solid substrate with detection probes immobilized thereon (e.g., chipannel 710) is optically clear with low auto fluorescence, for example, a glass with a high optical clarity such as borosilicate glass and soda glass.

In other embodiments, the solid substrate with detection probes immobilized thereon (e.g., chipannel 710) may be made of plastic materials high optical clarity. As non-limiting example, the substrate may be selected from the group consisting of polydimethylsiloxane (PDMS), cyclo-olefin copolymer (COC), polymethylmetharcylate (PMMA), polycarbonate (PC), cyclo-olefin polymer (COP), polvamide (PA), polyethylene (PE), polypropylene (PP), polyphenylene ether (PPE), polystyrene (PS), polyoxymethylene (POM), polyetheretherketonc (PEEK), polytetrafluoroethylene (PTFE), polvvinylchloride (PVC), polyvinylidene fluoride (PVDF), polyvinylalcohol, polyacylate, polybutyleneterephthalate (PBT), fluorinated ethylenepropylene (FEP), perfluoralkoxvalkane (PFA), polypropylene carbonate (PPC), polyether sulfone (PES), polyethylene terephthalate (PET), cellulose, poly(4-vinylbenzyl chloride) (PVBC), Toyopearlt, hydrogels, polyimide (PI), 1,2-polybutadiene (PB), fluoropolymers—and copolymers (e.g. poly(tetrafluoroethylene) (PTFE), perfluoroethylene propylene copolymer (FEP), Ethylene tetrafluoroethylene (ETFE)), polymers containing norbomene moieties, polymethylmethacrylate, acrylic polymers or copolymers, polystyrene, substituted polystyrene, polyimide, silicone elastomers, fluoropolymers, polyolefins, epoxies, polyurethanes, polyesters, polyethylene terephtalate, polypersulfone, and polyether ketones, and a combination thereof. The chips and chipannel may be prepared with injection mold.

In some embodiments, the substrate is optimized for immobilizing antibodies. The antibodies may be properly oriented on the surface without affecting the target binding activity. Techniques for the immobilization of antibodies may include physical and chemical immobilization. In some embodiments, antibodies may be directly immobilized onto solid surfaces by physical or chemical methods, for example, direct thiol-conjugating strategy. In other embodiments, antibodies may be printed onto solid surfaces through antibody-immobilizing linkers (e.g., Protein A/G and secondary antibodies).

In another embodiment of the test cup 300 shown FIG. 7E, the cup is further optimized to improve its performance and for manufacture. In this embodiment, the filter gasket 623 is overmolded to the interior of the cup body, e.g., in the homogenization chamber 321 (FIG. 7F). FIG. 7H demonstrates a cross-sectional view of the overmolded seal 713 that combines the parts into one single part. The overmolding facilitates the manufacturing process to result in a single piece. In this embodiment, the top of the valve shaft 351 of the rotary valve 350 comprises a cam 353 (FIG. 7G) that interacts with the filter cap 621 to provide a rotating motion (FIG. 7F, right panel). FIG. 7I demonstrates the cup bottom 337 (the top panel) and the bottom perspective view of the cup body 320 (the bottom panel). In this embodiment, the rotary valve 350 is secured in the test cup body 320 through a plurality of compression coil springs 721 located at the cup bottom cover 337 (FIG. 7J). FIG. 7J further demonstrates the compression coil springs 721 at the cup bottom 337. Four coil springs 721 may locate at the corners of the rotary valve port 350a to secure the valve 350. In this embodiment, the chipannel 710 may be welded to the bottom of the cup body 320. For example, the chipannel 710 may be laser welded to the bottom of the cup body 320. FIG. 7K demonstrates, in one example, the weld bead materials 722 at the bottom of the cup body 320 for laser welding.

The cup bottom 330 is configured to close the disposable test cup 300 and to provide a means for coupling the test cup 300 to the detection device 100 in various embodiments discussed herein. In some embodiments, the bottom side of the bottom assembly 330 of the cup 300 shown in FIG. 3H, includes several interfaces for connecting the cup 300 to the detection device 100 for operation, including a homogenization rotor interface 340a that may couple the homogenization rotor 340 to a motor in the device 100 for controlling homogenization: the valve interface 350a that may couple the rotary valve 350 to a motor in the device 100 for controlling valve rotation; and a pump interface 380 for connecting to a pump in the detection device 100.

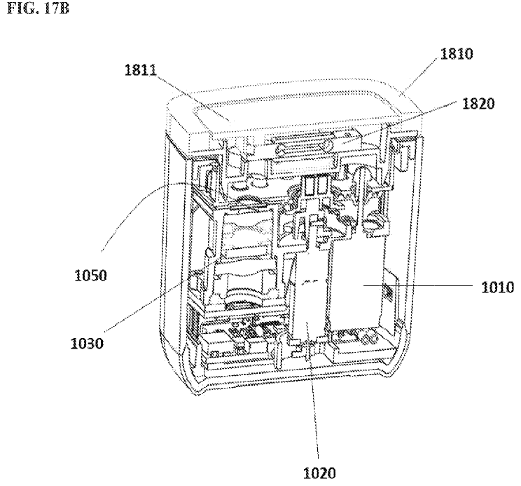

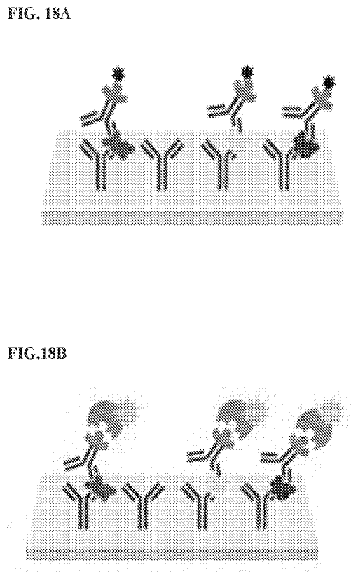

Another alternative embodiment of the analytical cartridge is provided in the present disclosure. The configuration of the test cup of this alternative embodiment is shown in FIG. 17A-17C, in which the test cup 1730 comprises a similar configuration of the compartments and other elements (e.g., shown in FIG. 7E) including a cup top or lid 1710, a cup body 320 that is configured to include a homogenization chamber 623, a filtrate chamber, wash chambers, a waste chamber, and a cup bottom 337.

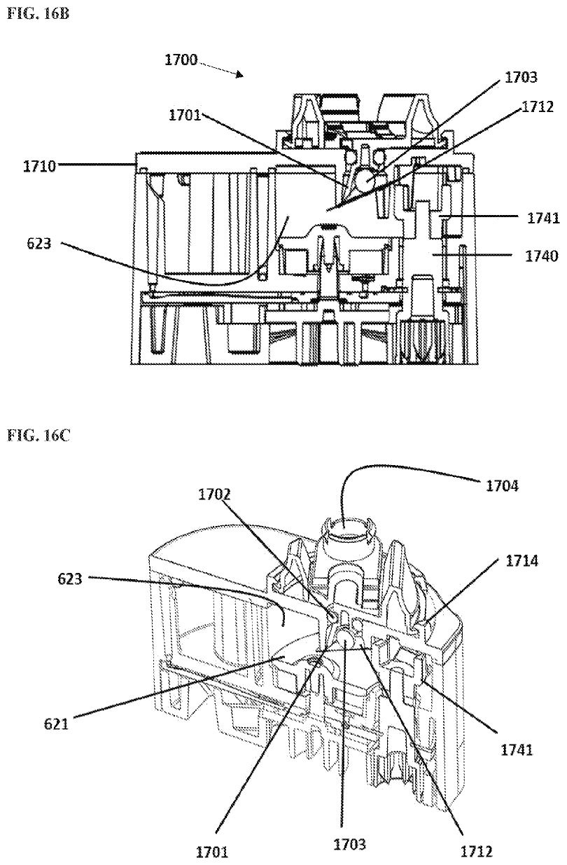

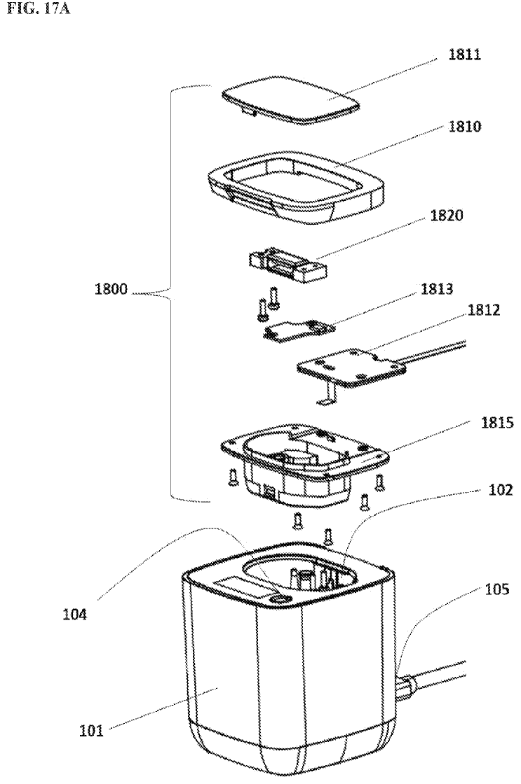

In this embodiment, the device includes a bead 1703 to assist or accelerate homogenization and detection. The bead may be made of any hard and non-reactive substance like metal, ceramic, or plastic. The bead may be smooth, textured, round, oblong, or any other shape to assist in homogenization. The bead may include or be coated with enzymes or other substances that assist in breaking down a sample. The embodiment includes a cap 1700 which may be removably attachable to the lid 1710 of the device. The cap 1700 includes at least one bead 1703 sealed within a pocket, defined between the cap 1700 and at least one seal, which may be foil or other film 1712. The seal 1712 is bonded to a lower portion of the lid 1710. The cap 1710 further includes a piercing member, such as a blade 1701. A cross section of the embodiment is shown in FIG. 16B demonstrating the juxtaposition of the blade 1701 to the foil and the homogenization chamber. The cap 1700 is securable in and movable within a groove 1714 or another track on the top of the lid 1710, with a compliant member 1711 separating the cap 1700 and the lid 1710. The lid 1710 has an opening 1715 through which the pocket and blade 1701 extend through. The cap 1700 may be rotated within the track 1714, between 1 degree to 360 degrees, about a central opening in the lid 1710, so that the blade 1701 pierces or slices through the seal, such as foil 1712. As the foil 1712 is opened, the bead is allowed to drop into the homogenization chamber. The cap 1700 also has an O-ring 1702, which creates a secure seal between the cap 1700 and the opening 1715 in the lid 1710. This secure seal allows the reaction chamber to be isolated from the ambient environment, even after the cap 1700 has been rotated or moved, cutting the seal and releasing the bead attached to the lid 1710.

The lid 1710 may include a food port 1716 through which a sampler 200 can deposit a sample. The food port 1716 may be covered and sealed by a port seal 1713. The port seal 1713 may be broken by the food corer 200, when depositing a food sample. The cap 1700 may have a port 1704 which aligns with the food port 1716 allowing the sampler 200 to pass through the cap 1700 and the lid 1710 and into the homogenization chamber 623. In operation, once the food has been deposited into the chamber 623, the cap 1700 is rotated in the groove 1714, which causes the blade 1701 to lance the foil 1712 and drop the bead 1703 into the chamber 623. The rotation of the cap 1700 to release the bead 1703 also causes the compliant member 1711 to rotate within the groove 1714 to cover and seal the food port 1716. The O-ring 1703 and the compliant member 1711 to securely seal the homogenization chamber 623.

The embodiment in FIG. 16A also includes a two-piece homogenization rotor including the rotor base 1740 and rotor blades 1741. Base 1740 and blades 1741 may be cold welded or compression-fit together. The rotor base 1740 engages with a drivetrain to spin the rotor blades 1741. The rotor base 1740 passes through the chipannel 710 and the rotor blades 1741 are attached to the top of the rotor base 1740; the rotor blades 1741 extend into the homogenization chamber 623. The rotor blades 1741 are configured to provide more power to the food sample to break up harder substances.

The embodiment in FIGS. 16A and 16B may also include a spacer 1722 instead of a gross filter 622, as included in the embodiment of 7E.

In some embodiments, a valve system is provided to control the fluid flow of the sample, detection agents, buffers and other reagents, and waste through different parts of the cartridge (e.g., separate chambers within the cup). In addition to flexible membranes, foil seals and pinch valves discussed herein, other valves may be included to control the flow of the fluid during the process of a detection assay, including swing check valves, gate valves, ball valves, globe valves, rotary valves, custom valves, or other commercially available valves. For example, a gland seal or rotary valve 350 may be used to control the flow of the processed sample solution within the cup 300. In some examples, pinch valves or rotary valves are used to completely isolate the fluid from other internal valve parts. In other examples, air operated valves (e.g., air operated pinch valves) may be used to control the fluid flow, which are operated by a pressurized air supply.

In one embodiment, means for controlling the fluid flow within the cup chambers may be included in, for example, the cup bottom assembly 330 and/or the cup body 320. The means may comprise flow channels, tunnels, valves, gaskets, vents, and air connections. In other embodiments, the means for the fluid flow may be configured as a separate component in the cup, e.g., the fluidic panel 631 shown in FIG. 6A.

In other embodiments, the valve system of the present disclosure may comprise additional air vents included in the test cup 300, to control air flow when the chip is used as the detection sensor. The sensor chip may be purged by air during the procession of an allergen detection assay. Individual air intakes may be opened based on the requirement of the system. The valve system as discussed herein may be used to keep the air vent unit inactive until use. The air port(s) allow air into the cartridge (e.g., the cup 300) and the air vent(s) allow air to enter various chambers when fluids are added to the chambers or removed from the chambers. The air vents may also have a membrane incorporated in them to prevent spillage and to act as a mechanism to control fluid fill volumes by occlusion of the vent membrane thus stopping further flow and fill function.

In one preferred embodiment, the rotary valve 350 (shown in FIG. 3C, FIG. 5B, FIGS. 6A and 6F and FIG. 7A) may be used to control and regulate fluid flow and rate in the test cup 300. The rotary valve 350 comprising a valve shaft 351 and a valve disc 352 (FIG. 6F and FIG. 7G) can be operated by an associated detection device (e.g., the device 100). In some embodiments, the rotary valve 350 may position at a specific angle by rotating the valve components either counterclockwise (CCW) or clockwise (CW) at each step of the repeated washing and air purge cycle(s) during the process of a detection assay. The air hole can allow air in. Air is drawn through the system via vacuum pressure to perform air purge functions. The angle may range from about 2° to about 75°.

As a non-limiting example, the valve may be at about 38.5° as reference from the air hole wherein the pump 1040 is off and the reaction chamber 331 is dry (referred to as home position). After the test sample is processed and homogenized, the pump is on and the valve 350 is rotated CCW and parks at an angle of about 68.5°, allowing the processed sample to be transported to the filtration chamber 322. Next, the valve components may be rotated again at different directions to park at different angles such as about 57° to flow wash buffer to the reaction chamber 331, and about 720 to purge the sensor chip with air. After the prewash of the sensor chip, the valve components may be rotated to the home position at about 38.5°. The processed sample solution is pulled through the filter assembly 325. After filtration, the valve components may be rotated and park at an angle of about 2°, allowing the collected filtrate to flow into the reaction chamber 331, wherein the chemical reactions occur. The valve 350 will rotate and park at about 57° to flow wash buffer to the reaction chamber 331, and park at about 72° to purge the sensor chip with air. The wash and air purge steps may be repeated one or more times until the optical measuring indicates a clean background.

In other embodiments, the rotary valve 350 is operatively connected to a filter cap 621 (FIG. 6E), the filter cap locks the rotary valve 350, for example during the shipment of the test cup 300.

In one embodiment, the valve system may be a rotary valve as shown in FIG. 8A and FIG. 8B. In this embodiment, the rotary valve 350 is positioned to control air in and fluid flow. The positioning may drive the homogenization in the homogenization chamber 321, filtration and collection of filtrates (F), sample washes (e.g., wash 1 (W1) and wash 2 (W2) and waste collection (in FIG. 8A). In step 1 of FIG. 8B, the rotary valve 350 is in a closed position with no connections being made between any of the chambers. In step 2 of FIG. 8B, the rotary valve 350 connects the wash 1 chamber W1 to the reaction chamber 331 to flush the reaction chamber 331 with the wash buffer subsequently being pushed out to the waste chamber 323. In step 3 of FIG. 8B, the rotary valve 350 connects the homogenization chamber 321 to the filtrate chamber F to affect the filtration step. In step 4 of FIG. 8B, the rotary valve 350 connects the filtrate chamber F to the reaction chamber 331 to send the filtrate to the reaction chamber 331 for reaction and analysis. In step 5 of FIG. 8B, the rotary valve 350 connects the wash 2 chamber W2 to the reaction chamber to flush the reaction chamber 331 again.

In some embodiments, extraction buffers may be pre-stored in the analytic cartridge, e.g., the homogenization chamber 321 of the cup body 320, for example in foil sealed reservoirs like the food processing reservoir 801 (FIG. 8C). Alternatively, extraction buffers may be stored separately in a separate buffer reservoir in the cup body 320, a reservoir similar to the wash buffer storage reservoir 802 (in the buffer storage chamber 324 (optional) as shown in FIG. 8C). The extraction buffer after sample homogenization and washing waste may be stored in the separate waste reservoir 803 within the waste chamber 323. The waste chamber 323 has sufficient volume to store a volume greater than the amount of fluid used during the detection assay.

In accordance with the present disclosure, the homogenization rotor 340 may be constructed to be small enough to fit into a disposable test cup 300, particularly into the homogenization chamber 321, where the homogenizer processes a sample to be tested. Additionally, the homogenization rotor 340 may be optimized to increase the efficacy of sample homogenization and protein extraction. In one embodiment, the homogenization rotor 340 may comprise one or more blades or the equivalent thereof at the proximal end. In some examples, the rotor 340 may comprise one, two, three or more blades. The homogenization rotor 340 is configured to pull the test sample from the food corer 200 into the bottom of the homogenization chamber 321.

Alternatively, the homogenization rotor 340 may further comprise a center rod running through the rotor that connects through the cup body 320 to a second interface bit. The central rod may act as an additional bearing surface or be used to deliver rotary motion to the rotor 340. When the rotor 340 is mounted to the cup body through the port at the cup bottom (e.g., 340a), the blade tips may remain submersed within the extraction buffer during operation. In another alternative embodiment, the homogenization rotor 340 may have an extension to provide a pass through the bottom of the cup; the pass may be used as a second bearing support and/or an additional location for power transmission. In this embodiment, the lower part of the rotor has a taper to fit to a shaft, forming a one-piece rotor. In accordance with the present disclosure, depth of the blades of the homogenization rotor 340, with or without the center rod, is constructed to ensure the blade tips in the fluid during sample processing.

As compared to other homogenizers (e.g., U.S. Pat. No. 6,398,402; incorporated herein by reference in its entirety), the custom blade core of the present disclosure spins and draws and forces food into the toothed surfaces of the custom cap. The homogenizer rotor may be made of any thermoplastics, including, but not limited to, polyamide (PA), acrylanitrilebutadienestyrene (ABS), polycarbonate (PC), high Impact polystyrene (HIPS), and acetal (POM).

The disposable cartridge may be in any shape, for example, circular, oval, rectangular, or egg-shaped. Any of these shapes may be provided with a finger cut or notch. The disposable cartridge may be asymmetrical, or symmetrical.

Optionally, a label or a foil seal may be included on the top of the cup lid 311 to provide a final fluid seal and identification of the test cup 300. For example, a designation of peanut indicates that the disposable test cup 300 is used for detecting the peanut allergen in a food sample.

### The Detection Device

In some embodiments, the detection device 100 may be configured to have an external housing 101 that provides support surfaces for the components of the detection device 100; and a lid 103 that opens the detection device 100 for inserting a disposable test cup 300 and covers the cup during operation. The small lid may be located at one side of the device (as shown in FIG. 1 and FIG. 9A), or in the center (not shown). In some aspects of the disclosure, the lid may be transparent, allowing all the operations visible through the lid 103. The device may also comprise s USB port 105 for transferring data.

One embodiment of the detection device 100 according to the present disclosure is depicted in FIG. 1 and FIG. 9A. As illustrated in FIG. 1, the detection device 100 comprising an external housing 101 that provides support for holding the components of the detection device 100 together. The external housing 101 may be formed of plastic or other suitable support material. In other embodiments, the device may be made of Aluminum. The device also has a port or receptacle 102 for docking the test cup 300 (FIG. 1 and FIG. 9A).

In some embodiments, the detection device 100 is provided with a means (e.g., a motor) for operating the homogenization assembly and necessary connectors that connect the motor to the homogenization assembly; means (e.g., a motor) for controlling the rotary valve; means for driving and controlling the flow of the processed sample solution during the process of a detection test; an optical system; means for detecting fluorescence signals from the detection reaction between the molecule of interest in the test sample and the detection agents; means for visualizing the detection signals including converting and digitizing the detected signals; a user interface that displays the test results; and a power supply.

As viewed from the transparent lid 103 (FIG. 9A), the device 100 has an interface comprising areas for coupling the components of the cartridge 300 (when inserted) for operating a detection reaction (FIG. 9B). These areas include a homogenization bit 910 for coupling the rotor 340 to the motor, a vacuum bit 920 for coupling the cup with the vacuum pump, a rotary valve drive bit 930 for coupling the rotary valve 350 to a valve motor and a protective glass 940 which is aligned to the chip 333 or the sensor area 333′ of the chipannel 710 through the optical window of the reaction chamber 331. A data chip reader 950 is also included to read the data chip 335. The pins 960 are used to facilitate placement of the cup 300 in the receptacle of the device 100.

In one embodiment of the present disclosure, as shown in FIG. 10A, the components of the detection device 100 that are integrated to provide all motion and actuation for operating a detection reaction, include a motor 1010 which may be connected to the homogenization rotor 340 inside the homogenization chamber 321 within the cup body 320. The motor 1010 may be connected through a multiple-component coupling assembly including a gear train/drive platen for driving the rotor during homogenization in an allergen detection test; a valve motor 1020 for driving the rotary valve 350; an optical system 1030 that is connected to the reaction chamber 331 (not shown) or the chipannel 710 within the disposable test cup 300; a vacuum pump 1040 for controlling and regulating air and fluid flow (not shown in FIG. 10A), a PCB display 1050, and a power supply 1060 (in FIG. 10B). A means for retaining the test cup (i.e., the cup retention 1070) is included for holding the test cup 300. Each part is described below in detail.

In one embodiment of the present disclosure, as shown in FIGS. 17A and 17B, the detection device 100 may include an integrated scale assembly 1800 as a lid for the device 100. The scale assembly may be operable to determine the weight, mass, or volume of the food sample to be analyzed in the device, prior to engaging the full assay of the device. By determining the weight of the food, a user can determine if the proper amount of food has been captured before turning on the assay. This prevents the waste of an assay, reagents, or a pod.

The integrated scale assembly 1800 may include a frame 1810 for connecting all the elements of the assembly 1800. A cover 1811 acts as the outer most lid of the device 100 as well as being operable as the scale surface onto which the food sample will be placed. The cover 1811 is supported by the frame 1810. The bottom of the cover 1811 rests on a strain gauge 1820 or other weight or mass sensing device. The strain gauge 1820 determines the weight of the food by parallel elements deflecting from a first neutral position. The extent of deflection of the elements allows for a measurable factor to be converted into weight. The processing may occur in electronics integrated within the integrated scale. The electronics within lid 1800 or within the housing 101 converts the analog output from the strain gauge 1820 to a digital output, rendering the weight. The strain gauge 1820 is supported by a gasket 1813 and a platform 1812, which may be operable to provide feedback to the strain gauge 1820 to assist in determining weight of the sample. The lid 1800 may draw power through a connection with the powered base 101 or the lid may have independent power and connectivity through the platform 1812. The lid 1800 and components therein are supported by the base 1815. It is within the conception of this embodiment that the lid 1800 includes other forms of scales or weight sensing devices, such as springs, load cells, laser vibrometry, accelerometer, driven coil, or other appropriately sized and configured weight measurement device. It is within the scope of this embodiment that multiple measurement device may be combined along with software to calculate or determine other characteristics of the sample, such as mass, volume, pH, density, hardness, moisture content, texture, or other factors useful for analysis and detection.

In operation, a user would place a portion of the test sample on the cover 1811 of the lid 1800. The sample exerts a force onto the cover 1811 and the strain gauge 1820 measures the displacement or the weight of the test sample. The lid with integrated scale 1800 may be integrated with the device 100 to give feedback to the system prior to beginning a detection assay. In order to prevent a wasted assay, software running the device would lock out the initiation of an assay until a sample of sufficient weight is measured. The lid 1800 is adapted to fit in the device 100, as shown in FIG. 10A, and integrate amongst the other components as shown in FIG. 17B. The frame 1810 including the strain gauge 1820 may fit within the receptacle 102. The optical system 1030 and display 1050 fit under the lid 1800. The homogenization motor 1010 and the valve motor 1020 have ample space in the device below the integrated lid 1800.

### 1. Homogenization Assembly

In one embodiment, the motor 1010 may be connected to the homogenization rotor 340 inside the test cup 300 through the multiple-component rotor coupling assembly. The rotor coupling assembly may include a coupling that is directly linked to the distal end cap of the rotor 340, and a gearhead that is part of a gear train or a drive (not shown) for connection to the motor 1010. In some embodiments, the coupling may have different sizes at each end, or the same sizes at each end of the coupling. The distal end of the coupling assembly may connect to the rotor 340 through the rotor port 340a at the cup bottom 330. It is also within the scope of the present disclosure that other alternative means for connecting the motor to the homogenization rotor 340 may be used to form a functional homogenization assembly.

In some embodiments, the motor 1010 can be a commercially available motor, for example. Maxon motor systems: Maxon RE-max and/or Maxon A-max (Maxon Motor ag, San Mateo, CA, USA).

Optionally, a heating system (e.g., resistance heating, or peltier heaters) may be provided to increase the temperature of homogenization, therefore, to increase the effectiveness of sample dissociation and shorten the processing time. The temperature may be increased to between 50° C. to 95° C., or 60° C. to 95° C., but below 95° C. Increased temperature may also facilitate the binding between detection molecules and the molecule being detected. Optionally a fan or peltier cooler may be provided to bring the temperature down quickly after implementing the test.

The motor 1010 drives the homogenization assembly to homogenize the test sample in the extraction buffer and dissociate/extract target proteins. The processed sample solution may be pumped or pressed through the flow tubes to next chamber for analysis, for example, to the reaction chamber 331 in which the processed sample solution is mixed with the pre-loaded detection agents for the detection test. Alternatively, the processed sample solution may first be pumped or pressed through the flow tubes to the filter assembly 325 and then to the filtrate chamber 322 before transported to the reaction chamber 331 for analysis.

### 2. Filtration

In some embodiments, means for controlling the filtration of the processed test sample may be included in the detection device. The test sample will be pressed through a filter membrane or a filtering assembly before the extraction solution being delivered to the reaction chamber 331, and/or other chambers for further processing such as washing. One example is the filter membrane(s). The membranes provide filtration of specific particles from the processed protein solution. For example, the filter membrane may filter particles up to from about 0.1 μm to about 1000 μm or about 1 μm to about 600 μm, or about 1 μm to about 100 μm, or about 1 μm to about 20 μm. In some examples, the filter membrane may remove particles up to about 20 μm, or about 19 μm, or about 18 μm, or about 17 μm, or about 16 μm, or about 15 μm, or about 14 μm, or about 13 μm, or about 12 μm, or about 11 μm, or about 10 μm, or about 9 μm, or about 8 μm, or about 7 μm, or about 6 μm, or about 5 μm, or about 4 μm, or about 3 μm, or about 2 μm, or about 1 μm, or about 0.5 μm, or about 0.1 μm. In one example, the filter membrane may remove particles up to about 1 μm from the processes sample. In some aspects, filter membranes may be used in combination to filter specific particles from the assay for analysis. This filter membrane may include multistage filters. Filter membranes and/or filter assemblies may be in any configuration relative to the flow valve. For example, the flow valves may be above, below or in between any of the stages of the filtration.

In some embodiments, the filter assembly may be a complex filter assembly 325 as illustrated in FIG. 4A in which the processed sample is filtered sequentially through the gross filter 411, the depth filter 412 and the membrane filter 420. In other embodiments, the filter assembly 325 may the filter stack shown in FIG. 6D.

### 3. Pump and Fluid Motion

In accordance with the present disclosure, a means for driving and controlling the flow of the processed sample solution is provided. In some embodiments, the means may be a vacuum system or an external pressure. As a non-limiting example, the means may be a platen (e.g., a welded plastic clamshell) configured to being multifunctional in that it could support the axis of the gear train and it could provide the pumping (sealed air channel) for the vacuum to be applied from the pump 1040 to the test cup 300. The pump 1040 may be connected to the test cup 300 through the pump port 920 located at the bottom (FIG. 9B), which connects to the pump interface 380 (FIG. 3G) on the bottom 330 of the test cup 300 when the cup is inserted to the device.

The pump 1040, such as piezoelectric micro pump (e.g., Takasago Electric, Inc., Nagoya, Japan), or peristaltic pump, may be used to control and automatically adjust the flow to a target flow rate. The flow rate of a pump is adjustable by changing either the driver voltage or drive frequency. As a non-limiting example, the pump 1040 may be a peristaltic pump. In another embodiment, the pump 1040 may be is a piezo pump currently on the market that have specifications that indicate they could be suitable for the aliquot function required to flow filtered sample solution to different chambers inside the test cup 300. The pump 1040 may be a vacuum pump or another small pump constructed for laboratory use such as a KBF pump (KNF Neuberger, Trenton, NJ, USA).

Alternatively, a syringe pump, diaphragm and/or a micro-peristaltic pump may be used to control fluid motion during the process of a detection assay and/or supporting fluidics. In one example, an air operated diaphragm pump may be used.

### 4. Rotary Valve Control

In some embodiments, the rotary valve 350 (e.g., as shown in FIG. 6F) for controlling fluid flow needs to be in precise positions. A means to control the rotary valve is provided and the control mechanism is able to rotate the valve in both directions and accurately stop at desired locations. In some embodiments, the device 100 includes a valve motor 1020 (in FIG. 10A). As shown in FIG. 11A, the valve motor 1020 may be a low cost, DC geared motor 1110 with two low-cost optical sensors (1131 and 1132), and a microcontroller. An output coupling 1120 interfaces with the rotary valve 350. In some embodiments, the output coupling 1120 has a ‘half-moon’ shelf 1170 as shown in FIG. 11B, which interrupts the output optical sensor 1131 with the protruding half. The output optical sensor signal toggles between high and low, depending on whether or not the protruding shelf interrupts the sensor. A microcontroller (MCU) detects these transitions and get an absolute position of the output from this signal. The positioning of these transitions is important and application specific since these transitions are used during directional changes to account for gear backlash.

The direct motor shaft 1140 has a paddle wheel which interrupts the direct shaft optical sensor 1132, allowing the direct shaft optical sensor 1132 to output a train of pulses, with the number of pulses per revolution determined by the number of paddles on the wheel 1150. The MCU reads this train of pulses and determines the degrees movement of the output coupling. The resolution is dependent on the number of paddles of the direct shaft encoder wheel 1150, and the gear reduction ratio of the gear box 1160.

The MCU interprets the output of these two optical sensors and can drive the output to a desired location, as long as the position of the output coupling shelf transitions, the number of paddle wheels on the direct encoder wheel 1120, and the gear ratio are known. During a change of direction, the motor must rotate by a fixed amount before an output transition is seen, the fixed amount is selected to overcome backlash of the gears. Once the fixed amount is overcome, on the next output signal transition, the MCU can start counting the direct signal pulses with confidence that they correspond to accurate output of location and movement.

### 5. Optical System

In practice, antibodies bind to proteins forming antibody-protein complexes. A secondary antibody (e.g., biotin-labeled secondary antibody). When the molecule to be tested is present in the sample, the antibody-protein complex can be detected with the secondary antibody. A fluorescently labeled streptavidin may be added to detect biotin. When bound to biotin, the fluorescence is detectable. The device and detection unit then presents the levels of fluorescence as positive or negative for the molecule of interest.

The detection device 100 of the present disclosure comprises an optical system that detects optical signals (e.g., a fluorescence signal) generated from the interaction between a molecule to be tested and detection agents (e.g., antibodies). The optical system may comprise different components and variable configurations depending on the types of the fluorescence signal to be detected. The optical system is close to and aligned with the detection cartridge, for instance, the primary optical window and optionally the secondary optical window of the reaction chamber 331 of the test cup 300 as discussed above.

In some embodiments, the optical system 1030 may include excitation optics 1210 and emission optics 1220 (FIGS. 12A and 12B). In one embodiment, as shown in FIG. 12A, the excitation optics 1210 may comprise a Light Emitted Diode (LED) 1211 configured to transmit an excitation optical signal to the sensing area (e.g., 332) in the reaction chamber 331, a collimation lens 1212 configured to focus the light from the light source, a filter 1213 (e.g., a bandpass filter), a focus lens 1214, and an optional LED power monitoring photodiode. The emission optics 1220 may comprise a focus lens 1221 configured to focus at least one portion of the allergen-dependent optical signal onto the detector (photodiode), two filters including a longpass filter 1222 and a bandpass filter 1223, a collection lens 1224 configured to collect light emitted from the reaction chamber and an aperture 1225. The emission optics collects light emitted from the solid surface (e.g., the sensor chip 333) in the detection chamber 331 and the signal is detected by the detector 1230 configured to detect an optical signal emitted from the sensing area 332. In some aspects, the excitation power monitoring may be integrated into the LED (not shown in FIG. 12A).

A light source 1211 is arranged to transmit excitation light within the excitation wavelength range. Suitable light sources include, without limitation, lasers, semi-conductor lasers, light emitting diodes (LEDs), and organic LEDs.

An optical lens 1212 may be used along with the light source 1211 to provide excitation source light to the fluorophore. An optical lens 1214 may be used to limit the range of excitation light wavelengths. In some aspects, the filter may be a band-pass filter.

In some embodiments, the emission optics 1220 are operable to collect emissions upon the interaction between detection agents and target allergens in the test sample from the reaction chamber 331. Optionally, a mirror may be inserted between the emission optics 1220 and the detector 1230. The mirror can rotate in a wide range of angles (e.g., from 1° to 90°) which could facilitate formation of a compacted optical unit inside the small portable detection device.

In some embodiments, more than one emission optical system 1220 may be included in the detection device. As a non-limiting example, three photodiode optical systems may be provided to measure fluorescence signals from an unknown test area and two control areas on a glass chip (e.g., see FIG. 13B). In other aspects, an additional collection lens 1224 may be further included in the emission optics 1220. This collection lens may be configured to detect several different signals from the chip 333. For example, when the detection assay is implemented using a sensor chip 333, more than two control areas may be constructed on the solid surface in addition to a detection area for target detection. The internal control signals from each control area may be detected at the same time when an allergen derived signal is measured. In this context, more than two collection lenses 1224 may be included in the optical system 1030, one lens 1224 for signal from the detection area and the remaining collection lenses 1224 for signals from the control areas.

The detector (e.g., photodiode) 1230 is arranged to detect light emitted from the fluidic chip in the emission wavelength range. Suitable detectors include, without limitation, photodiodes, complementary metal-oxide-semiconductor (CMOS) detectors, photomultiplier tubes (PMT), microchannel plate detectors, quantum dot photoconductors, phototransistors, photoresistors, active-pixel sensors (APSs), gaseous ionization detectors, or charge-coupled device (CCD) detectors. In some aspects, a single and/or universal detector can be used.

In some embodiments, the detector 1230 may be an image detector, such as a camera as described hereinbelow.

In some embodiments, the optical system 1030 may be configured to detect fluorescence signals from the solid substrate sensor (e.g., chip 333 shown in FIG. 13A or the chipannel 710 shown in FIGS. 7A to 7C). The sensor chip may be configured to contain a central reaction panel which is marked as an “unknown” signal area on the chip (FIG. 13A), and at least two control areas at various locations of the chip (FIG. 13A). In this context, the optical system 1030 is configured to measure both detection signals and internal control signals simultaneously (FIG. 13B).

In one example, the optical system 1030 comprises two collection lenses 1224 and corresponding optical components, such as control array photodiodes for each lens 1224. FIG. 12B demonstrates a side view of the optical system 1030 shown in FIG. 12A inside the detection device 100. In this embodiment, two collection lenses 1224 are included in the optical system, one for collecting control array signals from the sensor chip (e.g., the two signals 1301 and 1302 shown in FIG. 13B) and one specific to the unknown detection signal from the sensor chip (e.g., the detection signal 1302 as shown in FIG. 13B). In other aspects, the collection lenses 1224 may be configured to collecting signals from the detection area 333′ of the chipannel 710, e.g., one signal from the reaction panel 1312 and the other signal from the control panel 1313 shown in FIG. 13C. A signal array diode 1241 (e.g., the LED diode 1211 shown in FIG. 12A) and two control assay photodiodes 1242 are included for each optical path. Additionally, two prisms 1243 may be added to the two collection-lenses (1224) configured for collecting signals from the two control areas. The prisms 1243 can bend the control array light to the photodiode sensor area.

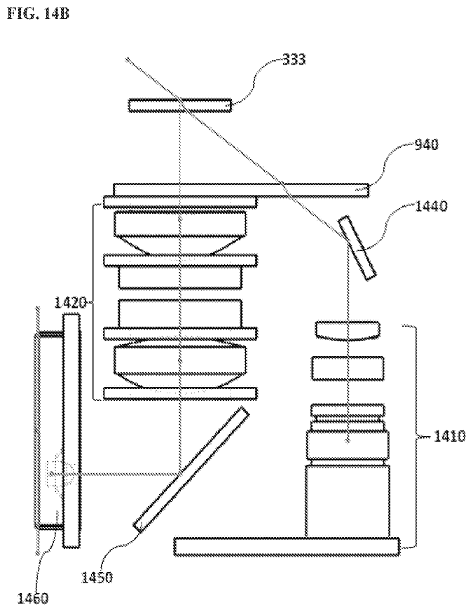

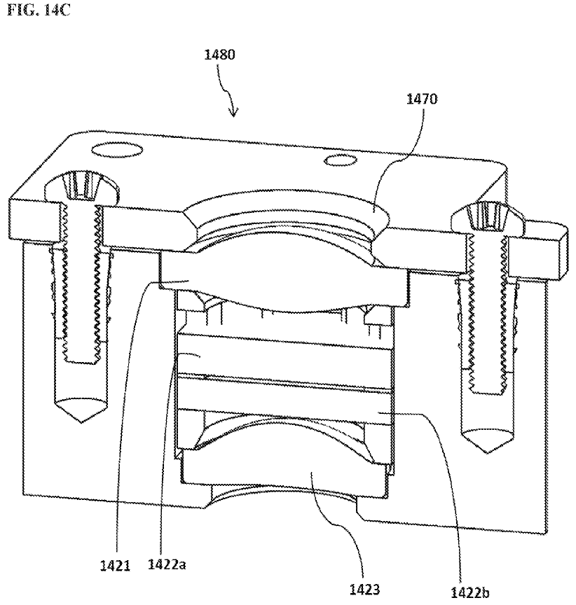

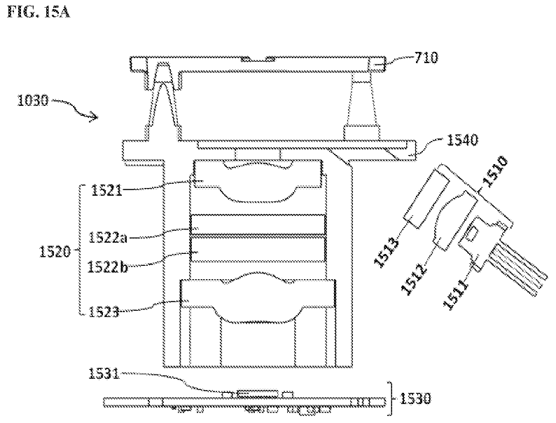

In some embodiments, the optical system 1030 may be configured as a straight mode as shown in FIG. 14A. The excitation optics 1410, which are configured to transmit an excitation optical signal to the chip 333 in the reaction chamber 331, may comprise a LED 1411, a collimation lens 1412, a bandpass filter 1413 and a cylinder lens 1414. The cylinder lens 1414 may cause the excitation light to form a line to cover the reaction panel and control panels on the glass chip (e.g., FIG. 13B). The emission optics 1420 which are aligned with the chip 333 may comprise a collection lens 1421 configured to collect light emitted from the chip 333, a bandpass filter 1422a, a longpass filter 1422b, and a focus lens 1423 configured to focus at least one portion of the allergen-dependent optical signal onto the chip reader 1430. The chip reader 1430 is composed of three photodiode lenses 1431, two control array photodiodes 1432, a signal array photodiode 1433 and a collection PCB 1434 (FIG. 14A). In some embodiments, the collection lens 1421 may be shaped to contain a concave first surface to optimize imaging and minimize stray light.

As a non-limiting example, the excitation optics 1410 and the emission optics 1420 may be folded and configured into a stepped bore 1480 in the device 100 (see FIG. 14C). An excitation folding mirror 1440 and a collection folding mirror 1450 may be configured to minimize the light paths from the excitation optics 1410 and the emission optics 1420, respectively (in FIG. 14B). The minimized volume can modulate the laser at a frequency to minimize interference from environmental light sources. A photodiode shield 1460 may be added to cover and protect the chip reader 1430 shown in FIG. 14A. The reader 1430 is then positioned close to the collection lens 1421 to minimize the scattered light. FIG. 14C illustrates an example of the stepped bore 1480 in the device to hold the emission optics 1420. The aperture 1470 of the collection lens 1421 is shown in FIG. 14C.

The LED source (e.g., 1411) may be modulated, and/or polarized and oriented to minimize the reflections from the glass chip. Accordingly, the chip reader may be synchronized to measure modulated light.

FIG. 15A illustrates another embodiment of the optical system 1030. In this embodiment, the optical system 1030 comprises an image detector. The image detector may be a camera 1531, as part of the signal reader 1530. The camera may catch the reaction images of the sensor chip 333 or the detection area 333′ of the chipannel 710. As a non-limiting example, the optical system 1030 shown in FIG. 15A, comprises an excitation optics 1510 comprising excitation filter 1513, collimation lens 1512 and laser diode 1511, an emission optics 1520 comprising a collection lens 1521, bandpass filter 1522a, longpass filter 1522b (e.g., color glass longpass filter) and focus lens 1523, and a signal reader 1530 comprising a camera 1531. Each system of the optical system may be configured in an optical housing, e.g., the optical housing 1540 in FIG. 15A configured for holding the components of the emission optics 1520.

FIG. 15B illustrates a cross-sectional view of the optical system of FIG. 15A assembled inside the detection device 100. From this cut-away side view, the excitation optics 1510 and the emission optics 1520 are assembled into an optical housing, respectively. A protective window 1501 may be added to protect the optical components. Optionally, a laser adjustment mount 1502 may be included to adjust the laser diode 1511 inside the excitation optics 1510. The camera 1531 catches the reaction images and the raw images are collected and processed. The detection results may be displayed through the display PCB 1050.

The above-described optical system 1030 is illustrative examples of certain embodiments. Alternative embodiments might have different configurations and/or different components.

In other embodiments, a computer or other digital control system can be used to communicate with the light filters, the fluorescence detector, the absorption detector and the scattered detector. The computer or other digital control systems control the light filter to subsequently illuminate the sample with each of the plurality of wavelengths while measuring absorption and fluorescence of the sample based on signals received from the fluorescence and absorption detectors.

### 6. Display

As shown in a cut-away side view in FIG. 10B, a printed circuit board (PCB) 1050 is connected to the optical system 1030. The PCB 1050 may be configured to be compact with the size of the detection device 100 and at the same time, may provide enough space to display the test result.

Accordingly, the test result may be displayed with back lit icons, LEDs or an LCD screen, OLED, segmented display or on an attached cell phone application. The user may see an indicator that the sample is being processed, that the sample was processed completely (total protein indictor) and the results of the test. The user may also be able to view the status of the battery and what kind of cartridge is placed in the device (bar code on the cartridge or LED assembly). The results of the test will be displayed, for example, as (1) actual number ppm or mg; or (2) binary result yes/no; or (3) risk analysis—high/medium/low or high/low, risk of presence; or (4) range of ppm less than 1/1-10 ppm/more than 10 ppm; or (5) range of mg less than 1 mg/between 1-10 mg/more than 10 mg. The result might also be displayed as number, colors, icons and/or letters.

In accordance with the present disclosure, the detection device 100 may also include other features such as means for providing a power supply and means for providing control of the process. In some embodiments, one or more switches are provided to connect the motor, the micropump and/or the gear train or the drive to the power supply. The switches may be simple microswitches that can turn the detection device on and off by connecting and disconnecting the battery.

The power supply 1060 may be a Li-ion AA format battery or any commercially available batteries that are suitable for supporting small medical devices such as the Rhino 610 battery, the Turntigy Nanotech High dischargeable Li Po battery, or the Pentax D-L163 battery.

In the description herein, it is understood that all recited connections between components can be direct operative connections or indirectly operative connections. Other components may also include those disclosed in the applicant's U.S. Provisional application 62/461,332, filed on Feb. 21, 2017; the contents of which are incorporated herein by reference in their entirety.

The allergen detection system may create a feedback loop for all stakeholders. The stakeholders may include a user, the user's family, caregivers, health care providers, or another party to whom data access is important (such as researchers). The system allows a user to input personal data into a user interface, such as on a smartphone. The system is then able to crowdsource data, which includes sharing the data to interested parties. The crowdsourcing may also allow for feedback in a consumer app, so that other users become aware of foods or restaurants that have a source of allergens or may be considered clear of the allergen. This access may assist interested users in deciding which foods, sources of foods, or restaurants may be considered safe from the allergens.

The system may create a neural network of users' feedback and results from certain detection tests. The neural network of data creates a competitive insulation to protect individual data if warranted, to alleviate HIPAA concerns. The crowdsourcing and neural networking of data creates a virtuous data loop beginning including 1) device software modifications, 2) food specifications from brands, 3) user food testing data, 4) leveraging of actionable information, and 5) algorithm improvements.

### Detection Assays

In another aspect of the present disclosure, provided is a detection test implemented using detection assemblies and systems, detection agents and detection sensors of the present disclosure.

As a non-limiting example, a detection test comprises the steps of (a) collecting and processing a test sample suspected of containing a molecule of interest; (b) contacting the processed sample with a detection sensor comprising a solid substrate that is printed with antibodies that specifically binds to the molecule of interest; (c) detecting the mixture in (b) and measuring fluorescence signals from the reaction; and (d) processing and digitizing the detected signals and visualizing the interaction between the detection agents and the target molecule.

In some aspects of the disclosure, the method further comprises the step of washing off the unbound compounds from the detection sensor to remove any non-specific binding interactions.

In some aspects of the disclosure, the method further comprises the step of filtering of the processed sample prior to contacting it with the detection sensor.

In some embodiments, an appropriately sized test sample is collected for the detection assay to provide a reliable and sensitive result from the assay. In some examples, a sampling mechanism that can collect a test sample effectively and non-destructively for fast and efficient extraction of allergen proteins for detection is used.

A sized portion of the test sample can be collected using, for example, a sampling corer 200 illustrated in FIG. 2B. The corer 200 collect an appropriately sized sample from which can be extracted sufficient protein for the detection test. The sized portion may range in mass from 0.1 g to 1 g, preferably 0.5 g. Furthermore, the corer 200 may pre-process the collected test sample by cutting, grinding, blending, abrading, and/or filtering. Pre-processed test sample will be introduced into the homogenization chamber 321 for processing and protein extraction.

The collected test sample is processed in an extraction/homogenization buffer. In some aspects, the extraction buffer is stored in the homogenization chamber 321 and may be mixed with the test sample by the homogenization rotor 340. In other aspects, the extraction buffer may be released into the homogenization chamber 321 from another separate storage chamber. The test sample and the extraction buffer will be mixed together by the homogenization rotor 340 and the sample being homogenized.

The extraction buffer may be universal target extraction buffer that can retrieve enough target proteins from any test sample and be optimized for maximizing protein extraction. In some embodiments, the formulation of the universal protein extraction buffer can extract the protein at room temperature and in minimal time (less than 1 min). The same buffer may be used during sampling, homogenization, and filtering. The extraction buffer may be PBS based buffer containing 10%, 20% or 40% ethanol, or Tris based buffer containing Tris base pH8.0, 5 mM MEDTA and 20% ethanol, or a modified PBS or Tris buffer. In some examples, the buffer may be a HEPES based buffer. Some examples of modified PBS buffers may include: P+ buffer and K buffer. Some examples of Tris based buffers may include Buffer A+, Buffer A, B, C, D, E, and Buffer T. As a non-limiting example, the extraction buffer may include 20 mM EPPS, 2% PEG 8000, 2% F-127 (Pluronic), 0.2% Brij-58 (pH8.4). In some embodiments, the extraction buffer may be optimized for increasing protein extraction. A detailed description of each modified buffer is disclosed in the PCT Patent Application No.: PCT/US2014/062656; the content of which is incorporated herein by reference in its entirety.

The volume of the extraction buffer may be from 0.5 mL to 3.0 mL. In some embodiments, the volume of the extraction buffer may be 0.5 mL, 1.0 mL, 1.5 mL, 2.0 mL, 2.5 mL, or 3.0 mL. The volume has been determined to be efficient and repeatable over time and in different matrices (e.g., food, clinical samples, environmental samples and veterinary samples).

In accordance with the present disclosure, the test sample is homogenized and processed using the homogenization assembly that has been optimized with high speed homogenization for maximally processing the test sample.

In some aspects of the disclosure, a filtering mechanism may be linked to the homogenizer. The homogenized sample solution is then driven to flow through a filter in a process to further extract allergen proteins and remove particles that may interfere with the flow and optical measurements during the test, lowering the amount of other molecules extracted from the test sample. The filtration step may further achieve uniform viscosity of the sample to control fluidics during the assay. In the context that DNA glass chips are used as detection sensors, the filtration may remove fats and emulsifiers that may adhere to the chip and interfere with the optical measurements during the test. In the context that chips printed with antibodies are used as detection sensors, the filtration may reduce non-specific bindings. In some embodiments, a filter membrane such as cell strainer from CORNING (CORNING, NY, USA) or similar custom embodiment may be connected to the homogenizer. The filtering process may be a multi-stage arrangement with different pore sizes from first filter to second, or to the third. The filtering process may be adjusted and optimized depending on matrices being tested. As a non-limiting example, a filter assembly with a small pore size may be used to capture particles and to absorb large volumes of liquid when processing dry foods, therefore, longer times and higher pressures may be used during the filtration. In another example, bulk filtration may be implemented to absorb fat and emulsifiers when processing fatty foods. The filtration may further facilitate to remove fluorescence haze or particles from fluorescence samples, which will interfere with the optical measurements.

The filter may be a simple membrane filter, or an assembly composed of a combination of filter materials such as PET, cotton, and sand, etc. In some embodiments, the homogenized sample may be filtered through a filter membrane, or a filter assembly, e.g., the filter assembly 325 in FIG. 4A.

In some aspects of the present disclosure, the sampling procedure may reach effective protein extraction in less than 1 minute. In one aspect, speed of digestion may be less than 2 minutes including food pickup, digestion, and readout. Approximately, the procedure may last 15 seconds, 30 seconds, 45 seconds, 50 seconds, 55 seconds, 1 minute or 2 minutes.

The interaction between protein extraction and detection agents will generate a detectable signal which indicates the presence, or absence or the amount of one or more targets in the test sample. As used herein, the term “detection agent” refers to any molecule which is capable of, or does, interact with and/or bind to one or more targets in a way that allows detection of such target in a sample. The detection agent may be a protein-based agent such as antibody, a nucleic acid-based agent, or a small molecule.

In some embodiments, the detection agent is an antibody. As used herein, the term “antibody” is used in the broadest sense and specifically includes (but is not limited to) whole antibodies, monoclonal antibodies, polyclonal antibodies, multispecific antibodies (e.g., bispecific antibodies formed from at least two intact antibodies), antibody fragments, diabodies, antibody variants, and antibody-derived binding domains that are part of or associated with other peptides. Antibodies are primarily amino acid based molecules but may also comprise one or more modifications (including, but not limited to the addition of sugar moieties, fluorescent moieties, chemical tags, etc.)

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen.

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein the term, “antibody fragment” refers to any portion of an intact antibody. In some embodiments, antibody fragments comprise antigen binding regions from intact antibodies. Examples of antibody fragments may include, but are not limited to Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Compounds and/or compositions of the present invention may comprise one or more of these fragments. For the purposes herein, an “antibody” may comprise a heavy and light variable domain as well as an Fc region.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012, the contents of which are herein incorporated by reference in their entirety.

As used herein, the term “antibody variant” refers to a biomolecule resembling an antibody in structure and/or function comprising some differences in their amino acid sequence, composition or structure as compared to a native antibody.

In some embodiments, the interaction between protein and antibodies are detected by the secondary antibody. The secondary antibody may be labeled with a fluorescence marker. The fluorescence marker, fluorophore may suitably have an excitation maximum in the range of 200 to 700 nm, while the emission maximum may be in the range of 300 to 800 nm. The fluorophore may further have a fluorescence relaxation time in the range of 1-7 nanoseconds, preferably 3-5 nanoseconds. As non-limiting examples, a fluorophore that can be probed at one terminus of a SPN may include derivatives of boron-dipyrromethene (BODIPY, e.g., BODIPY TMR dye; BODIPY FL dye), fluorescein including derivatives thereof, rhodamine including derivatives thereof, dansyls including derivatives thereof (e.g. dansyl cadaverine), texas red, eosin, cyanine dyes, indocarbocyanine, oxacarbocyanine, thiacarbocyanine, merocyanine, squaraines and derivatives seta, setau, and square dyes, naphthalene and derivatives thereof, coumarin and derivatives thereof, pyridyloxazole, nitrobenzoxadiazole, benzoxadiazole, anthraquinones, pyrene and derivatives thereof, oxazine and derivatives, nile red, nile blue, cresyl violet, oxazine 170, proflavin, acridine orange, acridine yellow, auramine, crystal violet, malachite green, porphin, phthalocyanine, bilirubin, tetramethylrhodamine, hydroxycoumarin, aminocoumarin; methoxycoumarin, cascade blue, pacific blue, pacific orange, NBD, r-phycoerythrin (PE), red 613; perCP, trured; fluorX, Cy2, Cy3, Cy5 and Cy7, TRITC, X-rhodamine, lissamine rhodamine B, allophycocyanin (APC) and Alexa fluor dyes (e.g., Alexa Fluo 488, Alexa Fluo 500, Alexa Fluo 514, Alexa Fluo 532, Alexa Fluo 546, Alexa Fluo 555, Alexa Fluo 568, Alexa Fluo 594, Alexa Fluo 610, Alexa Fluo 633, Alexa Fluo 637, Alexa Fluo 647, Alexa Fluo 660, Alexa Fluo 680, and Alexa Fluo 700).

In some embodiments, the secondary antibody is labeled biotin. In this embodiment, the signal can be detected using fluorescently labeled streptavidin.

In some embodiments, detection agents for eight major food allergens (i.e., wheat, egg, milk, peanuts, tree nuts, fish, shellfish, and soy) may be provided as disposables. In one aspect, constructs of the detection agents may be stored with MgCl₂, or buffer doped with KCl. MgCl₂ keeps constructs closed tightly, while KCl opens them slightly for bonding.

In some embodiments, the detection sensor is a solid substrate printed with antibodies. As used herein, the term “detection sensor” refers to an instrument that can capture a reaction signal, i.e., the reaction signal derived from the binding of target proteins and antibodies, measure a quantity and/or a quality of a target, and convert the measurement to a signal that can be measured digitally.

In some embodiments, the detection sensor is a solid substrate, such as a chip, on which antibodies are immobilized (FIGS. 18A-B). For example, the detection sensor may be the chip 333 inserted into the reaction chamber 331 of the present disclosure or a chipannel 710 in the test cup 300 (FIG. 7A). The detection sensor may also be a separate glass chip, for example, prepared from glass wafer and soda glass, or a microwell, or an acrylic glass, or a microchip, or a plastic chip made of COC (cyclic olefin copolymer) and COP (cyclo-olefin polymer), or a membrane like substrate (e.g., nitrocellulose), of which the surface is coated with antibodies.

In some embodiments, the antibody coated chip may comprise at least one reaction panel and at least two control panels. The reaction panel is printed with antibodies that bind to the target proteins. The control panels are printed with other antibodies that do not bind to target proteins (referred herein as “control antibodies”). In some examples, the control antibodies are labeled with a fluorescence marker.

In some embodiments, the sample is processed in the homogenization chamber 321. The target protein, if present in the test sample, when the sample solution flows to the detection sensor, e.g., the chip 333 in the reaction chamber 331 (FIG. 3B) or the chipannel 710 (FIG. 7A), will bind to the antibodies. The protein:antibody complex can be detected using fluorescently labeled secondary antibodies. A fluorescence signal will be detected from the reaction panel (as shown in FIGS. 13A and 13B).

In some embodiments, antibody probes may be printed to a reaction panel at the center of a glass chip (“unknown”) and control antibodies may be printed to the two control panels at each side of the reaction panel on the chip, as illustrated in FIG. 13A.

In some embodiments, the sensor chip may be prepared by any known protein printing technologies known in the art. In some embodiments, the chip may be prepared by using single spot pipetting to pipette antibody solution onto the glass chip, or by stamping with a wet PDMS stamp comprising an antibody solution followed by pressing the stamp against the glass slide, or by flow with microfluidic incubation chambers.

As a non-limiting example, a glass wafer can be laser cut to produce 10×10 mm glass “chips”. Each chip contains three panels: one reaction panel (i.e., the “unknown” area in the chip demonstrated in FIG. 13A) that is flanked by two control panels (FIG. 13A). The reaction panel contains antibodies. In a detection assay, the reaction panel of the chip faces a small reaction chamber (e.g., the reaction chamber 331) flanked by an inlet and outlet channel (e.g., 336 in FIG. 3H) of the cartridge (e.g., the cup 300). The processed sample solution including the extracted proteins enters the reaction chamber 331 via the inlet, through fluidic movement driven by a vacuum pump. The solution then exits into a waste chamber 323 via the outlet channel. After exposure to the sample, the reaction panel is then washed, revealing a fluorescence signal with an intensity correlated to the target protein concentration.

In some embodiments, the wash buffer is optimized to improve wash efficiency, increasing baseline signal and decreasing non-specific binding. As a non-limiting example, the wash buffer may be an optimized PPB buffer, including pluronic F-127 (e.g., 2% w/v), PEG-8000 (2% w/v), Brij 58 (e.g., 0.2% w/v) and EPPS (e.g., 20 mM), pH8.4.

In accordance with the present disclosure, the two control panels are constantly bright areas on the chip sensor that produce a constant signal as background signals 1301 and 1302 (FIG. 13B). In addition, the two control panels compensate for laser illumination and/or disposable cartridge misalignment. If the cartridge is perfectly aligned, then the fluorescence background signals 1301 and 1302 would be equal (as shown in FIG. 13B). If the measured control signals are not equal, then a look-up table of correction factors will be used to correct the unknown signal as a function of cartridge/laser misalignment. The final measurement is a comparison of the signal 1303 of the unknown test area against the signal levels of the control areas. The comparison level may be one of the lot-specific parameters for the test.

Samples with high background fluorescence measurements from the reaction area may produce a false negative result. A verification method may be provided to adjust the process.

The final fluorescence measurement of the reaction panel, after being compared to the controls and any lot specific parameters may be analyzed and a report of the result may be provided.

Accordingly, the light absorption and light scattering signals may also be measured at the baseline level, before and/or after the injection of the processed sample. These measurements will provide additional parameters to adjust the detection assay. For example, such signals may be used to look for residual sample in the reaction chamber 331 after wash.

In addition to the parameters discussed above, one or more other lot-specific parameters may also be measured. The optimization of the parameters, for example, may minimize the disparity in the control and unknown signal levels for the chips.

In some embodiments, the monitoring process may be automatic and is controlled by a software application. Evaluation of the sensor chip and test sample, the washing process and the final signal measurement may be monitored during the detection assay.

### Applications

The detection systems, devices and methods described herein contemplate for detection of any molecule of interest in a sample (e.g., a food sample, a clinical sample and a veterinary sample). The portable devices allow a user to test the presence or absence of the target molecule in the testing sample.

In some embodiments, an allergen can be detected in a food sample. Allergen families that can be detected using the device described herein include allergens from legumes such as peanuts, tree nuts, eggs, milk, soy, spices, seeds, fish, shellfish, wheat gluten, rice, fruits and vegetables. The allergen may be present in a flour or meal. The device is capable of confirming the presence or absence of these allergens as well as quantifying the amounts of these allergens. Allergen families that can be detected using the detection system and device described herein include allergens from foods, the environment or from non-human proteins such as domestic pet dander. Food allergens include, but are not limited to proteins in legumes such as peanuts, peas, lentils and beans, as well as the legume-related plant lupin, tree nuts such as almond, cashew, walnut, Brazil nut, filbert/hazelnut, pecan, pistachio, beechnut, butternut, chestnut, chinquapin nut, coconut, ginkgo nut, lychee nut, macadamia nut, nangai nut and pine nut, egg, fish, shellfish such as crab, crawfish, lobster, shrimp and prawns, mollusks such as clams, oysters, mussels and scallops, milk, soy, wheat, gluten, corn, meat such as beef, pork, mutton and chicken, gelatin, sulphite, seeds such as sesame, sunflower and poppy seeds, and spices such as coriander, garlic and mustard, fruits, vegetables such as celery, and rice. The allergen may be present in a flour or meal, or in any format of products. For example, the seeds from plants, such as lupin, sunflower or poppy can be used in foods such as seeded bread or can be ground to make flour to be used in making bread or pastries.

The system includes science and technology controls with built-in precision and sensitivity controls with both the assay and any hardware feedback look being responsive to the user and consumer touchpoints. An application or other software control (such as on a smartphone) provides a detailed tutorial on usage of the system and, in conjunction with a guide and website, illustrates consumer use recommendations. Customer support ensures support for the product and user. The system follows all performance testing methods and recommendations from AOAC and other governing bodies related to allergy and allergen detection as well as publish independent lab verification. The system may include a carrying case to hold the device, extra cartridges or pods, as well as other related devices such as an epinephrine, diphenhydramine tables, or other emergency medicines. The application may help decrease the risk of encountering an allergen.

The system, as a whole, has an ease of functionality which ensures user success. The user first collects a food sample and can weigh the sample on an integrated scale in the lid of the detection device as discussed above. The system can alert the user if additional food is needed and will indicate that the pd should be removed and to add more food. The system will also indicate if too much food has been added to the pod and indicate that a new pod should be used. Once enough food has been added and processed, the system ensures that the resultant images are analyzable. After an image analysis has been generated, the system determines if the image meets acceptance criteria. After this point, the system provides results-whether the allergen (a peanut, etc.) is detected or not detected. A control panel in the system (i.e., on a smartphone) indicates how the food impacts the assay regardless of the presence of peanut and can be used to calibrate the test panel system. An algorithm of the system incorporates the intensity of the test and control panel, in addition to the time of reaction, background, and pressure curves to yield the most accurate output. The algorithm utilizes a stringent penalty function to skew output away from false negatives.

The pod is installed in the device and monitored for optical and fluidic connection. The rotor or homogenizer speed is monitored during food mastication or processing. The flow and mixing step is monitored via an on-board pressure transducer. Minimal imaging standards are assessed prior to result reporting.

In a broad concept, the detection systems, devices and methods described herein may be used for detection of any protein content in a sample in a large variety of applications in addition to food safety, such as, for example, medical diagnosis of diseases in civilian and battlefield settings, environmental monitoring/control and military use for the detection of biological weapons. In even broad applications, the detection systems, devices, and methods of the present disclosure may be used to detect any biomolecules. As some non-limiting examples, the detection systems, devices and methods may be used on the spot detection of cancer markers, in-field diagnostics (exposure the chemical agents, traumatic head injuries etc.), third-world applications (TB, HIV tests etc.), emergency care (stroke markers, head injury etc.) and many others.

As another non-limiting example, the detection systems, devices, and methods of the present disclosure can detect and identify pathogenic microorganisms in a sample. Pathogens that can be detected include bacteria, yeasts, fungi, viruses and virus-like organisms. Pathogens cause diseases in animals and plants; contaminate food, water, soil, or other sources; or is used as biological agents in military fields. The device is capable of detecting and identifying pathogens.

Another important application includes the use of the detection systems, devices, and methods of the present disclosure for medical care, for example, to diagnose a disease, to stage a disease progression and to monitor a response to a certain treatment. As a non-limiting example, the detection device of the present disclosure may be used to test the presence or absence, or the amount of a biomarker associated with a disease (e.g., cancer) to predict a disease or disease progression. The detection systems, devices and methods of the present disclosure are constructed to analyze a small amount of test sample and can be implemented by a user without extensive laboratory training.

Another important application includes the use of the detection systems, devices, and methods of the present disclosure for veterinary cares.

Other expanded applications outside of the field of food safety include in-field use by military organizations, testing of antibiotics and biological drugs, environmental testing of products such as pesticides and fertilizers, testing of dietary supplements and various food components and additives prepared in bulk such as caffeine and nicotine, as well as testing of clinical samples such as saliva, skin and blood to determine if an individual has been exposed to significant levels of an individual allergen.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

A number of possible alternative features are introduced during the course of this description. It is to be understood that, according to the knowledge and judgment of persons skilled in the art, such alternative features may be substituted in various combinations to arrive at different embodiments of the present disclosure.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. An assembly for detecting a molecule of interest in a sample comprising:
a sample processing cartridge having a homogenization chamber configured to accept the sample for processing to a state permitting the molecule of interest to engage in an interaction with a detection agent; the cartridge comprising:
a lid,
a movable cap, the movable cap having a lancing element,
a transparent substrate comprising the detection agent, and
a homogenization accelerator, which is secured in a pocket bounded by the cap and a seal on the lid;
a detector unit configured to accept the sample processing cartridge in a configuration which permits a detection mechanism housed by the detector unit to detect the interaction of the molecule of interest with the detection agent, wherein the interaction triggers a visual indication on the detector unit that the molecule of interest is detected; and
wherein the visual indication is by processing images capturing the interaction of the molecule of interest with the detection agent.

2. The assembly of claim 1 wherein the movable cap is rotatably secured to the lid; and
wherein the lid further comprises at least one aperture opening into the homogenization chamber.

3. (canceled)

4. The assembly of claim 2 wherein the pocket is co-located with the at least one aperture.

5. The assembly of claim 4 wherein movement of the movable cap causes the lancing element to lance the seal allowing the homogenization accelerator to enter the homogenization chamber.

6. The assembly of claim 2 wherein the at least one aperture further includes a second aperture opening into the homogenization chamber.

7. The assembly of claim 6 wherein the cap further includes a port which, in a first position of the cap, co-localizes with the second aperture; the second aperture containing a breakable seal facing the homogenization chamber.

8. The assembly of claim 7 wherein in a second position of the cap the second aperture is covered by the cap and sealed by a movable cover.

9. (canceled)

10. The assembly of claim 1 wherein the detection agent is an antibody or variant thereof, a nucleic acid molecule or variant thereof, or a small molecule.

11. (canceled)

12. The assembly of claim 1 wherein the sample processing cartridge comprises:
a homogenizer configured to produce a homogenized sample, thereby releasing the molecule of interest from a matrix of the sample into an extraction buffer;
a plurality of separate chambers including the homogenization chamber, a filtrate chamber, and a detection chamber;
a first conduit to transfer the homogenized sample through a filter system to provide a filtrate containing the molecule of interest; and
a second conduit to transfer the filtrate to a detection chamber with a window;
wherein the detection mechanism of the detector unit analyzes the detection chamber through the window to identify the interaction of the molecule of interest with the detection agent in the detection chamber.

13. The assembly of claim 12 wherein the homogenizer comprises a rotor and wherein the rotor is powered by a motor located in the detector unit, wherein the motor is functionally coupled to the homogenizer when the sample processing cartridge is accepted by the detector unit, and wherein the homogenization accelerator is configured to engage with the rotor to assist in homogenization.

14. The assembly of claim 12 wherein the sample processing cartridge further comprises a chamber holding wash buffer for washing the detection chamber and a waste chamber for accepting outflow contents of the detection chamber after wash.

15. The assembly of claim 14 wherein the sample processing cartridge further comprises a rotary valve system for controlling transfer of the homogenized sample to the filter system, for transfer of the filtrate to the detection chamber, for transfer of the wash buffer to the detection chamber and for transfer of contents of the detection chamber to the waste chamber.

16. The assembly of claim 15 wherein the rotary valve system is further configured to provide a closed position to prevent fluid movement in the sample processing cartridge.

17. The assembly of claim 12 wherein the detection chamber includes the transparent substrate with the detection antibody and control antibodies immobilized thereon;
wherein the transparent substrate further comprises a fluidic panel in connection with the detection antibody for transfer of the filtrate containing the molecule of interest to contact with the detection antibody and control antibodies; and
wherein the assembly further comprises an assembly lid capable of measuring the weight, mass, or volume of a sample.

18. (canceled)

19. (canceled)

20. The assembly of claim 17 wherein the assembly lid further comprises:
a frame,
a base attached to the frame, and
a cover connected to the frame; wherein the cover includes a measurement device adjacent thereto and above the base, whereby the measurement device is capable of detecting and measuring the weight, mass, or volume of the sample when the sample is placed on the cover.

21. The assembly of claim 20 wherein the measurement device is a strain gauge or a load gauge.

22. The assembly of claim 21 further comprising a sampler, the sampler comprising a hollow tube with a cutting edge for cutting a source to generate and retain the sample within the hollow tube and a plunger for pushing the sample out of the hollow tube and into a port in the sample processing cartridge, the sampler capable of breaking the seal on the second aperture.

23. A system for detecting the presence or absence of a molecule of interest in a sample, comprising:
a sampler for collecting a sample suspected of containing the molecule of interest;
a disposable analytical cartridge configured for processing the sample, thereby permitting the molecule of interest in the sample to engage in the interaction with a detection agent; and
a detection device configured for measuring the sample, operating the detection test, and measuring and visualizing a signal from the binding interaction between the detection agent and the molecule of interest presented in the sample.

24. The system of claim 23 wherein the disposable analytical cartridge comprises:
a lid with a housing and a movable cap, the movable cap having a lancing element, and a homogenization accelerator, which is secured in a pocket bounded by the cap and a seal on the lid;
a sample processing chamber with a homogenizer configured to homogenize the sample with an extraction buffer, thereby permitting the molecule of interest in the sample to engage in the interaction with a detection agent;
a filter system configured to provide a filtrate containing the molecule of interest and the detection agent;
a separate transparent substrate comprising a plurality of fluidic channels and a detection area with the detection agent immobilized thereon;
a detection chamber with an optical window;
a chamber holding wash buffer for washing the substrate and the detection chamber;
a waste chamber for accepting and storing outflow contents of the detection chamber after wash;
a rotary valve system and conduits configured to transfer the homogenized sample and detection agent through the filler system, to transfer the filtrate to the detection chamber, and to transfer the wash buffer to the detection chamber and outflow contents from the detection chamber to the waste chamber; and
an air flow system configured to regulate air pressure and flow rate in the cartridge.

25. (canceled)

26. (canceled)

27. The system of claim 23 wherein the detection device comprises:
a frame attachable to the housing;
a base attached to the frame; and
a cover connected to the frame; and wherein the cover includes a measurement device adjacent thereto and above the base, whereby the measurement device is capable of detecting and measuring the weight, mass, or volume of the sample when the sample is placed on the cover.
